SEQUENCE LISTING (10 sequences)

SEQ ID NO: 1 - DNA, 2139 bp, Homo Sapiens
ctggtcttcaagagcgatgcctattactgcactggggacgtcactgcctggaccaagtgtatggtcaagacacagacacccaaccggaaggagtgggtaaccccaaaggaattccgagaaatctcttacctcaagaaattgaaggttaaaaagcaggaccgtatattccccccagaaaccagcgcctccgtggcggccacgcctccgccctccacagcctcggctcctgctgctgtgaactcctctgcttcagcagataagccattatccaacatgaagatcctgactctcgggaagctgtcccggaacaaggatgaagtgaaggccatgattgagaaactcggggggaagttgacggggacggccaacaaggcttccctgtgcatcagcaccaaaaaggaggtggaaaagatgaataagaagatggaggaagtaaaggaagccaacatccgagttgtgtctgaggacttcctccaggacgtctccgcctccaccaagagccttcaggagttgttcttagcgcacatcttgtccccttggggggcagaggtgaaggcagagcctgttgaagttgtggccccaagagggaagtcaggggctgcgctctccaaaaaaagcaagggccaggtcaaggaggaaggtatcaacaaatctgaaaagagaatgaaattaactcttaaaggaggagcagctgtggatcctgattctggactggaacactctgcgcatgtcctggagaaaggtgggaaggtcttcagtgccacccttggcctggtggacatcgttaaaggaaccaactcctactacaagctgcagcttctggaggacgacaaggaaaacaggtattggatattcaggtcctggggccgtgtgggtacggtgatcggtagcaacaaactggaacagatgccgtccaaggaggatgccattgagcagttcatgaaattatatgaagaaaaaaccgggaacgcttggcactccaaaaatttcacgaagtatcccaaaaagttttaccccctggagattgactatggccaggatgaagaggcagtgaagaagctcacagtaaatcctggcaccaagtccaagctccccaagccagttcaggacctcatcaagatgatctttgatgtggaaagtatgaagaaagccatggtggagtatgagatcgaccttcagaagatgcccttggggaagctgagcaaaaggcagatccaggccgcatactccatcctcagtgaggtccagcaggcggtgtctcagggcagcagcgactctcagatcctggatctctcaaatcgcttttacaccctgatcccccacgactttgggatgaagaagcctccgctcctgaacaatgcagacagtgtgcaggccaaggtggaaatgcttgacaacctgctggacatcgaggtggcctacagtctgctcaggggagggtctgatgatagcagcaaggatcccatcgatgtcaactatgagaagctcaaaactgacattaaggtggttgacagagattctgaagaagccgagatcatcaggaagtatgttaagaacactcatgcaaccacacacagtgcgtatgacttggaagtcatcgatatctttaagatagagcgtgaaggcgaatgccagcgttacaagccctttaagcagcttcataaccgaagattgctgtggcacgggtccaggaccaccaactttgctgggatcctgtcccagggtcttcggatagccccgcctgaagcgcccgtgacaggctacatgtttggtaaagggatctatttcgctgacatggtctccaagagtgccaactactaccatacgtctcagggagacccaataggcttaatcctgttgggagaagttgcccttggaaacatgtatgaactgaagcacgcttcacatatcagcaggttacccaagggcaagcacagtgtcaaaggtttgggcaaaactacccctgatccttcagctaacattagtctggatggtgtagacgttcctcttgggaccgggatttcatctggtgtgatagacacctctctactatataacgagtacattgtctatgatattgctcaggtaaatctgaagtatctgctgaaactgaaattcaattttaagacctccctgtgg

SEQ ID NO: 2 - protein, 713 aa, Homo Sapiens
LVFKSDAYYCTGDVTAWTKCMVKTQTPNRKEWVTPKEFREISYLKKLKVKKQDRIFPPETSASVAATPPPSTASAPAAVNSSASADKPLSNMKILTLGKLSRNKDEVKAMIEKLGGKLTGTANKASLCISTKKEVEKMNKKMEEVKEANIRVVSEDFLQDVSASTKSLQELFLAHILSPWGAEVKAEPVEVVAPRGKSGAALSKKSKGQVKEEGINKSEKRMKLTLKGGAAVDPDSGLEHSAHVLEKGGKVFSATLGLVDIVKGTNSYYKLQLLEDDKENRYWIFRSWGRVGTVIGSNKLEQMPSKEDAIEQFMKLYEEKTGNAWHSKNFTKYPKKFYPLEIDYGQDEEAVKKLTVNPGTKSKLPKPVQDLIKMIFDVESMKKAMVEYEIDLQKMPLGKLSKRQIQAAYSILSEVQQAVSQGSSDSQILDLSNRFYTLIPHDFGMKKPPLLNNADSVQAKVEMLDNLLDIEVAYSLLRGGSDDSSKDPIDVNYEKLKTDIKVVDRDSEEAEIIRKYVKNTHATTHSAYDLEVIDIFKIEREGECQRYKPFKQLHNRRLLWHGSRTTNFAGILSQGLRIAPPEAPVTGYMFGKGIYFADMVSKSANYYHTSQGDPIGLILLGEVALGNMYELKHASHISRLPKGKHSVKGLGKTTPDPSANISLDGVDVPLGTGISSGVIDTSLLYNEYIVYDIAQVNLKYLLKLKFNFKTSLW

SEQ ID NO: 3 - DNA, 23 bp, Artificial Sequence
  Primer
aagtcgacgctggtcttcaagag

SEQ ID NO: 4 - DNA, 22 bp, Artificial Sequence
  Primer
aactcgagccaccgggaggtct

SEQ ID NO: 5 - DNA, 28 bp, Artificial
  Primer
aaagaattcttccgagaaatctcttacc

SEQ ID NO: 6 - DNA, 28 bp, Artificial Sequence
  Primer
aaactcgagttccacatcaaagatcatc

SEQ ID NO: 7 - DNA, 28 bp, Artificial Sequence
  Primer
aaagaattcagtatgaagaaagccatgg

SEQ ID NO: 8 - DNA, 26 bp, Artificial Sequence
  Primer
aaactcgagccacagggaggtcttaa

SEQ ID NO: 9 - DNA, 20 bp, Artificial Sequence
  Primer
tcaagcttactatccttgtc

SEQ ID NO: 10 - DNA, 20 bp, Artificial Sequence
  Primer
tcaagcttactatccttgtc

(19) United States
(12) Patent Application Publication
(10) Pub. No.: US 2004/0180385 A1
(43) Pub. Date: Sep. 16, 2004

(54) Method of diagnosing systemic lupus erythematosus

(76) Inventors: Dooil Jeoung, Seoul (KR); Saeyoung Park, Incheon (KR); Daeyeon Lee, Incheon (KR); Hosoon Lee, Seoul (KR); Mina Lee, Kyunggi (KR); Seongeun Lee, Seoul (KR); Myungin Baek, Seoul (KR); Bomsoo Cho, Seoul (KR); Yoon Lim, Seoul (KR); Jongwan Kim, Seoul (KR); Kyuchoon Hwang, Seoul (KR); Yung-Jue Bang, Seoul (KR); Han-Kwang Yang, Seoul (KR); Yeongwook Song, Seoul (KR); Dae-Kee Kim, Seoul (KR); Eunbong Lee, Seoul (KR); Joungyeon Kim, Seoul (KR)

Correspondence Address: JHK Law, P.O. Box 1078, La Canada, CA, 91012-1078, US

(21) Appl. No.: 10/388,929

(22) Filed: Mar. 14, 2003

(51) Int. Cl. G01N 33/53

(52) U.S. Cl. 435/7.9

(57) ABSTRACT

The application discloses a method of diagnosing an autoimmune disease in a mammal comprising: a) obtaining an antibody-containing sample from a subject suspected of suffering from the autoimmune disease, b) contacting said sample with a composition comprising PARP polypeptide; and c) detecting the presence of PARP polypeptide antigen/anti-PARP polypeptide antibody complex, which is indicative of the presence of the autoimmune disease.

### BACKGROUND OF THE INVENTION

1. Field of the Invention

The present invention relates to the identification of autoantibodies specifically associated with systemic lupus erythematosus (SLE). The present invention concerns the use of specific antigen to detect the presence of CF2K-specific antibodies in an individual suspected of having SLE, wherein the presence of such antibodies is indicative of said individual suffering from SLE.

2. General Background and State of the Art:

Autoimmune diseases are characterized by an abnormal immune response (involving either immune system cells or antibodies) directed against normal self-tissues. It afflicts huge numbers of individuals throughout the world. A common characteristic of autoimmune diseases is the presence of one or more types of antinuclear antibodies (ANA) in the body fluids of patients suffering from the disease. ANA's are autoantibodies directed against antigens in the nucleus and cytoplasm of a person's own cells. Many of these autoantibodies are used as markers for autoimmune diseases. In the majority of connective tissue disorders, the relationship between the pathogenesis and the presence of autoantibody has not been clearly resolved. Autoimmune diseases are multisystemic, and their heterogeneous nature makes early and definitive diagnosis a big challenge. Even with the availability of disease-specific autoantibodies, a clear diagnosis of these diseases requires a number of different assays. Examples of autoimmune disorders are numerous and include, but not limited to, systemic lupus erythematosus, rheumatoid, arthritis, Addison's disease, Sjogren syndrome, Behcet disease, Graves' disease, Crohn's disease, autoimmune aplastic anemia, Celiac disease, alopecia totalis, giant cell arteries, insulin-dependent diabetes mellitus, juvenile rheumatoid arthritis, Lambert-Eaton syndrome, multiple sclerosis, narcolepsy, neonatal lupus syndrome, psoriasis, Reiter's syndromne, stiff-man syndrome, myocarditis, polymyositis, primary sclerosing cholangitis, and ulcerative colitis.

Systemic lupus erythematosus (SLE), commonly known as lupus, is an autoimmune disease characterized by dysregulation of the immune system resulting in the production of antinuclear antibodies, the generation of circulating immune complexes, and the activation of the complement system. The immune complexes build up in the tissues and joints causing inflammation, and degradation to both joints and tissues. SLE affects the entire body and most organ systems; the disease often involves inflammation and consequent injury to the joints, skin, kidney, brain, the membranes in body cavities, lung, heart, and gastrointestinal tract. Clinical manifestations of SLE are so varied that it bears a great deal of similarity to a plethora of other autoimmune diseases including rheumatoid arthritis and polymyositis, amongst others. This heterogeneity has necessitated the use of a list of diagnostic criteria to be fulfilled before a definitive diagnosis of the disease can be attained. There are at least 14 criteria that can be examined; if four or more of these criteria are present then SLE is indicated. These criteria include, facial erythema, discoid lupus rash, Raynaud's phenomenon, alopecia, photosensitivity, oral, nasal or pharyngeal ulceration, arthritis without deformity, LE cells, false positive tests for syphilis, proteinurea, pleuritis, pericarditis, psychosis, convulsions, hemolytic anemia, leukopenia, and thrombocytopenia.SLE is not a rare disease. Although reported in both the extremely old and the extremely young, the disease is chiefly found in women of childbearing age. Among children the occurrence of SLE is three times more likely in females than in males. In the 60% of the SLE patients who experience the onset of this disease between puberty and the fourth decade of life, the female to male ratio is 9:1. Thereafter, the female ponderance again falls to that observed in prepubescent children (i.e. 3:1). In addition, the disorder appears to be three times more common in persons of African and Asian descent than in persons of Caucasian descent. The prevalence of SLE in the United States is an issue of some debate. Estimates of occurrence range from 250,000 to 2,000,000 persons.

Problems with identifying SLE are part of the problem in providing estimates of the numbers of individual affected. The etiology of SLE remains unknown. A genetic predisposition, the systemic proliferation of sex hormones, and an environmental trigger likely result in the disordered immune response that typifies the disease. A genetic basis for SLE in human is complex, with an unknown but non-Mendelian mode of inheritance. This complexity has impeded the development of a reliable and predictive genetic test.

Many investigators have reported that certain human MHC class II alleles (HLA-DR and/or DQ but not DP) and certain class III genes (C2, C4, TNF-alpha and Hsp70-2 alleles) confer susceptibility to SLE in most ethnic groups studied. Among the other non-MHC genes that have been associated with SLE, evidence for homozygous deficiency of C1q predisposing to SLE is particularly compelling, including the observation that 90% of such individuals have SLE, and C1q knockout mice display an SLE-like phenotype (M. Botto et al., Nat. Genet. 19: 56-59, 1998). In addition, polymorphisms in many genes encoding molecules with relevant immunological functions have been studied most frequently by the case-control approach, including T-cell receptor alpha and beta chains, immunoglobulin allotypes, FcyRIIa, FcgRIIIa, IL-6, IL-10, Bcl-2, mannose-binding protein, as well as deletion of specific variable gene segments of immunoglobulin genes (F. C. Arnett Jr., 1997; T. J. Vyse and B. L. Kotzin, 1998, J. Wu et al., 1998, A novel polymorphism of FcγR1ilA, which alters function, associates with SLE phenotype, J. Invest. Med. 45: 200A; R. Mehrian et al., Synergistic effect between IL-10 and Bcl-2 genotypes in determining susceptibility to systemic lupus erythematosus, Arthritis Rheum. 41: 596-602, 1998). Mutations in nucleic acids encoding T cell receptor zeta chain have been linked to SLE in some patients (K. Tsuzaka et al., 1998, J. Autoimmun. 11(5): 381-385). Some candidate genes may confer risk only to subsets of SLE patients. For example, FcγR1IA alleles confer an increased risk of lupus GN in African Americans, but not in Caucasians, or persons of Afro-Caribbean or Chinese origin (J. E. Salmon et al., J. Clin. Invest. 97: 1348-1354, 1996). In addition, there is an increased frequency of the extended haplotypes HLA-A1, B8, and DR3 in affected individuals. The role for heredity is further supported by the concordance for this illness among monozygotic twins. There is evidence for a corresponding genetic linkage in human SLE. Using the identified murine susceptibility loci (the overlapping SLE/Nba2/Lbw7) as a guide, Tsao et al. examined seven markers located on a syntenic human chromosomal 1q31-942 region, corresponding to the telomeric end of mouse chromosome 1, the latter being the region where contributions to specific manifestations of murine lupus, including glomerulonephritis and IgG anti-chromatin, have been mapped (B. Tsao et al., 1997, J. Clin. Invest. 99: 725-731).

The body's immune system normally makes proteins called autoantibodies to protect the body against viruses, bacteria and other foreign materials. These foreign materials are called antigens. In an autoimmune disorder, the immune system loses its ability to tell the difference between antigens and its own cells and tissues. The immune system then makes antibodies directed against “self”. These antibodies, called, “autoantibodies”, react with the “self autoantigens” to form immune complexes. The immune complexes build up in the tissues, causing inflammation, injury to tissues, and pain. There is no specific treatment for SLE, but several drugs are known to modify the disease so that the symptoms are tolerable. These treatment regimens include non-steroidal anti-inflammatory drugs (NSAIDs) and analgesic; steroids, such as, prednisolone and methylprednisolone, hydroxychloroquine, azathioprine and cyclophosphamide. The potential benefit of these treatments is counterbalanced by adverse side effects, which are almost inevitable. One of the most common causes of death in SLE patients is secondary infection. Although the causes of SLE are unknown, certain factors definitely exacerbate the symptoms of the disease; these include UV light and certain hormones. Such factors can be avoided using barried creams and avoiding contraceptives containing estrogens.

Systemic lupus erythematosus (SLE) is an autoimmune disease associated with autoantibodies directed against cell nuclei, membrane, and cytoplasm (E. M. Tan et al., Adv. Immunol. 1989, 44:93-151). These so-called antinuclear autoantibodies can be divided into three groups: 1. True antinuclear antigens (ANA): directed against dsDNA, ssDNA, histones, nucleolic RNA, and DNP, 2. Extractable nuclear antigens (ENA): Sm (Smith), n-RNP, Scl70, Jo-1 and pM-1,3. Cytoplasmic antigens: SS-A (Ro) ad SS-B (La). Regardless of the clinical manifestations of the disease, patients with SLE almost invariably express anti-nuclear antibodies. Such antibodies also are produced in most other rheumatic disease, are produced transiently in viral infections and are present, usually in low titers, in about two percent of the normal population.

The best-investigated immunoreactive antigens are double stranded DNA, single stranded DNA, Sm (Smith), sn-RNP (small nuclear ribonucleoprotein particles), the complex RNP/Sm that is stabilized by ribonucleic acid as well as SS-A (Ro) and SS-B (La). Of the many anti-nuclear antibodies produced, two are considered diagnostic for SLE, namely, anti-double stranded DNA antibodies and antibodies to Sm. Anti-ds DNA antibodies bind sites on the helical backbone of the native DNA. The anti-Sm binds to proteins on an RNA-protein complex termed Sn RNP (small nuclear ribonucleoprotein complex). It is thought that antinuclear antibodies are responsible for the tissue injuries associated with autoimmune disease. Thus, the detection of these antinuclear antibodies can be of significant clinical or diagnostic value. Antinuclear antibodies (ANA) are prominent in the biological fluids of patients suffering from disorders such as Systemic lupus erythematosus (SLE), Systemic sclerosis (SSc), mixed connective tissue disease (MCTD), and Sjogren's syndrome.

ANAs are targeted against soluble and particulate nucleoproteins of both double- and stranded single stranded DNA, single-stranded and double-stranded RNA as well as a saline extractable nuclear constituent (Sm antigen). Additionally, autoantibodies have been found against the mitochondria, ribosomes, lysosomes, a soluble cytoplasmic fraction, red cells, white cells, platelets and blood clotting factors. Immunofluorescence (IF) staining of cultured human cell subtrates is useful in determining the presence of antinuclear antibodies in human sera but do not provide information regarding the specificities of the antibodies. The disease specificity of well-characterized antinuclear antibodies is an important diagnostic aid when interpreted in conjunction with the patient's clinical condition. It can also be used to partition patients into clinical subsets, and in some instances the presence or titer of a specific antinuclear antibody correlates with the patient's prognosis.

The use of IF staining is still the standard method used to screen for the presence of antinuclear antibody. If there are antibodies in the patient's serum that are immunoreactive with antigen components associated with the cell, they will bind to the cells and form an antigen-antibody complex. The presence of a fluorescence signal is detected by viewing the cells under fluorescent microscope. The fluorescent signal is transient and will disappear within a few hours or days. Disease severity, and in some cases response to therapy, is measured by increases and decreases in the antinuclear antibody titer in a patient.

In addition to determining the presence or absence of fluorescent signal, detection of certain patterns of fluorescence also provides useful information in diagnosing the specific disease that may be afflicting a patient. For example, a homogeneous fluorescent nuclear signal is indicative of SLE, while a speckled pattern can indicate Scleroderma, MCTD, Sjogren's syndrome or Raynaud's syndrome. The staining pattern is due to the reactivity of the patient's antibodies to specific nuclear or cytoplasmic components.

The IF staining methods are not strictly diagnostic of specific autoimmune disease because of the substantial overlap of the fluorescent patterns exhibited by a number of autoimmune diseases. Also, if the sample is not titered appropriately, masking of fluorescent patterns can occur. Thus, IF staining is tedious method, which does not generate a permanent record, involves multiple assays, is time-consuming and labor-intensive, and requires considerable expertise in the interpretation of results.

Western blots (immunoblots) have been used to detect certain antinuclear antibody specificities. The use of immunoblots for the detection of specific antigens recognized by antinuclear antibody provides several advantages over IF, immunodiffusion, immunoprecipitation, and EIA methods including a) more complete information can be obtained about the number and molecular size of the antigen c) immunoblot is sensitive and reproducible. The antigen specificity of the antibody can be determined by comparison of the sample reactivity with control markers on the blot. However, the Western blot method cannot be used to detect antibodies that are immunoreactive with double stranded DNA since membranes that bind proteins do not bind DNA well unless the transfer occurs under alkaline conditions. Western blot cannot handle a large number of samples at the same time.

In this invention, we carried out SEREX (serological analysis of recombinant cDNA expression library) to identify autoantibodies associated with SLE. SEREX combines both molecular and immunological approaches to identify molecules associated with various diseases. The advantage of using SEREX is that it enables us to identify molecules, on a genomic scale, that are specifically associated with disease of choice.

SEREX (Serological identification of antigens by recombinant Expression cloning) methodology has been used to identify genes encoding human tumor antigens (Gure et al., Proc. Natl. Acad. Sci., 97: 4198-4203 (2000); Soiffer et al., Proc. Natl. Acad. Sci., 95: 13141-13146 (1998); Gure et al., Cancer Research, 58:1034-1041 (1998); and Scanlan et al., Int. J. Cancer, 76: 652-658 (1998)) or CTLs (Boon et al., J. Exp. Med., 183:725-729 (1996); Wolfel et al., Science, 260: 1281-1284 (1995); Gnjatic et al., J. Immunol. 160, 328-333 (1998); Brandle et al., J. Exp. Med., 183: 2501-2508 (1996); Coulie et al., J. Exp. Med., 180: 35-42 (1994); and Van den Eynde et al., J. Exp. Med., 182:689-698 (1995)). Examples of tumor antigens that are recognized by antibodies include GM2 (Livingston et al., Proc. Natl. Acad. Sci., 84: 2911-2915 (1987)), Her/neu (Disis et al., Cancer Research, 54: 16-20 (1994)), and p53 (Labrecque et al., Cancer Research, 53: 3468-3471 (1993). In the SEREX method, autologous serum is used to detect cancer-specific antigens that have immunogenicity. Genes that have been identified by SEREX include those that are over-expressed (Disis et al., Cancer Research, 54: 16-20 (1994)), mutated (Wolfel et al., Science, 260: 1281-1284 (1995); and Robbins et al., J. Exp. Med., 183: 1185-1192 (1996)), alternatively spliced, differentiated (Coulie et al., J. Exp. Med., 180: 35-42 (1994)), and those that are specifically expressed in cancer and normal testis (Chen et al., Proc. Natl. Acad. Sci., 94: 1914-1918 (1997); Chen et al., Proc. Natl. Acad. Sci., 95:6919-6923 (1998); Gure et al., Int. J. Cancer, 72: 965-971 (1997); and Martelange et al., Cancer Res., 60: 3848-3855 (2000)). SEREX has been applied to a variety of tumors, including melanoma, esophageal cancer, renal cancer, astrocytoma, and colon cancer. Many of these SEREX antigens are specifically expressed in cancer and normal testis.

There are several known C/T (cancer/testis) antigens including MAGE (Gauge et al., J. Exp. Med., 179: 921-930 (1994)), BAGE (Boel et al., Immunity, 2: 167-175 (1995)), and NY-ESO-1 (Jager et al., J. Exp. Med., 187: 265-270 (1998)). These antigens were originally identified in melanoma. It was reported that MAGE and BAGE showed higher expression in metastatic melanoma than in primary melanoma. This indicates that selective expression of C/T antigen is associated with dedifferentiation. Identification of these antigens is necessary for diagnosis and therapeutic development. Although it is highly sensitive, SEREX is not suitable for analysis of large series of sera because of extensive serum adsorptions. ELISA would be more suitable for analysis of large series of sera of patients with SLE.

### SUMMARY OF THE INVENTION

It is an object of this invention to identify an autoimmune disease-specific antibody, such as SLE (systemic lupus erythematosus)-specific autoantibody. In preferred embodiment, the identification of SLE-specific serologic marker can be carried out by SEREX (serological analysis of recombinant cDNA expression library) technique. It is also an object of this invention to determine the frequency of this autoantibody in various rheumatic diseases, including, but not limited to, rheumatoid arthritis, Sjogern disease, myositis, and systemic sclerosis.

It is an object of this invention to provide a method for producing a protein antigen, which is reactive with an autoantibody associated with autoimmune disease. It is an object of this invention to provide a method for readily providing a protein antigen reactive with an autoantibody associated with autoimmune disease. It is an object of this invention to construct various domains of PARP protein. It is an object of this invention to produce polypeptides comprising various domains of PARP protein or PARP polypeptide. It is another object of this invention to provide a method capable of producing such antigen in large amounts and without requiring donation of large amounts of material from a subject. It is an object of this invention to determine sensitivity and specificity of such protein for the diagnosis of SLE. It is an object of this invention to provide a method of diagnosing an autoimmune disease in a human comprising the steps of obtaining an antibody-containing sample; contacting the sample with a composition comprising a PARP polypeptdie antigen, such as but not limited to CF2K, which is a fragment of PARP; and detecting the presence of a PARP polypeptide/anti-PARP polypeptide antibody complex; wherein the presence of a PARP polypeptide antigen/anti-PARP polypeptide antibody complex is diagnostic for an autoimmune disease such as SLE.

In preferred embodiments, the sample tested may be blood, plasma, serum or any other test sample employed in diagnostic assays. In another embodiments, the detecting comprises the use of a technique such as ELISA, RIA, immunoprecipitation, or Western immunoblot. In a further embodiment, detecting is carried out by ELISA. In preferred embodiment, ELISA comprises the steps of providing a preparation comprising a PARP polypeptideK antigen bound to support; contacting the preparation with the sample whereby a PARP polypeptide antigen/anti-PARP polypeptide antibody complex is formed; and contacting the complex with a detection agent. It is contemplated that the detection agent is an anti-Fc antibody that binds the anti CF2K antibody. In preferred embodiments, the antibody may be labeled with a label selected from the group consisting of an enzyme, radiolabel, biotin, a dye, a hapten, luminescent label, and a fluorescent tag label. The enzyme may be alkaline phosphatase or horseradish peroxidase. In certain embodiments, the fluorescent tag can be selected from the group consisting of fluorescein, luciferase, green fluorescent protein, and rhodamine. In another embodiment, the dye may be phycocyanin, phycoerythrin, texas red, or o-phthaldehyde.

In another embodiment, the support may be any material that is well-known in the art, for example a microtiter plate, a polystyrene bead, test tube or dipstick. It is an object of this invention to provide a kit comprising a PARP polypeptide antigen preparation, and a suitable container means therefor. The kit may contain secondary antibody preparation that detects PARP polypeptide antigen/PARP polypeptide-antibody complex that is present in the biological samples of persons suspected of having an autoimmune disease such as SLE. In another embodiment, the secondary antibody comprises a detectable label. The detectable label may be a radiolabel, an enzyme, biotin, a dye, a fluorescent tag, a hapten, or a luminescent label. In those embodiments in which the label is an enzyme, the kit may further comprise a substrate for the enzyme. The kit may contain other components including reagent reservoirs, instruction manual, and the like, which are well known to those of skill in the art. It is also an object of this invention to test currently available diagnostic kit for the detection of systemic lupus erythematosus.

These and other objects of the invention will be more fully understood from the following description of the invention, the referenced drawings attached hereto and the claims appended hereto.

### DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows seroreactivity of patient with SLE for PARP. K-ZAP phages without insert were mixed with test clones and served as negative control. Assays were scored positive only when test clones were clearly distinguishable from control phages. Bold arrow indicates test clone and blank arrow indicates control clone.

FIG. 2 shows functional domains of PARP protein. Catalytic domain of PARP contains NAD⁺-binding sites. The numbers denote amino acid residues.

FIG. 3 shows functional domains inserted into expression vectors. Each PCR product (a domain of PARP) was subcloned into appropriate restriction sites of pET21 vector. F1, F2 denote zinc finger domains.

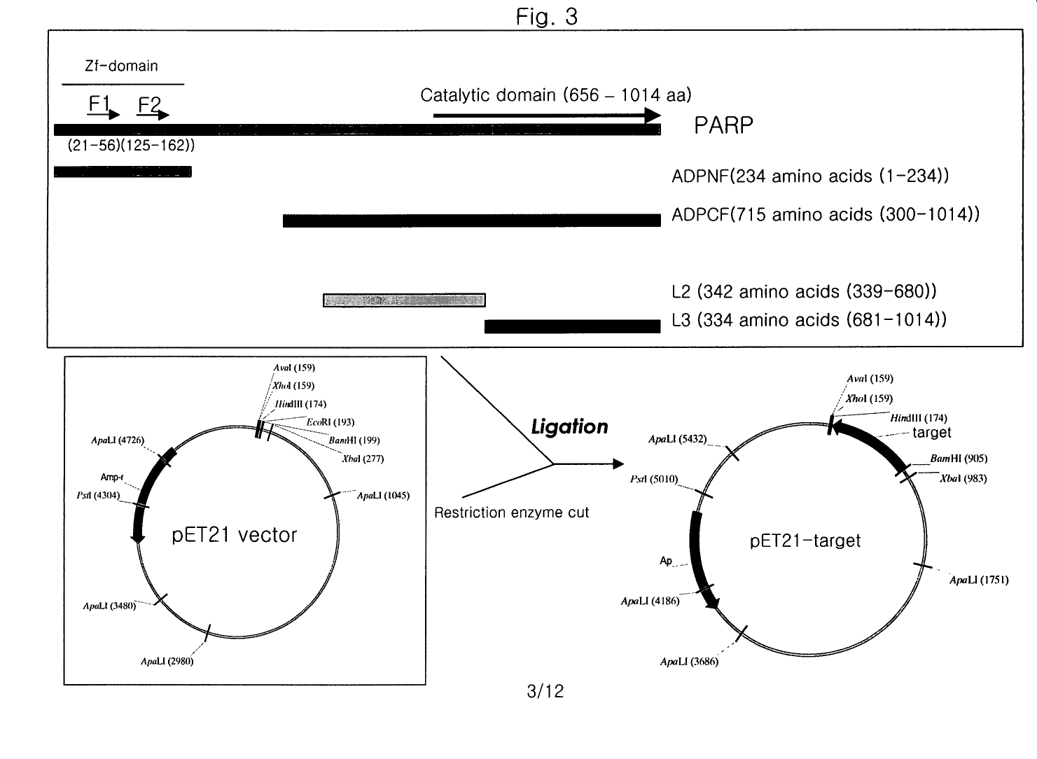

FIG. 4 shows induction and purification of ADPNF protein. The identity of eluates was confirmed by Western immunoblot using either anti-His antibody or anti-PARP antibody. E denotes eluate. W denotes washing fraction, and F denotes flow-through fraction. Control lane was loaded with cell lysates prepared from E. coli cells transformed with control vector (without insert).

FIG. 5 shows induction and purification of ADPCF2K protein. The identity of eluates was confirmed by Western blot analysis using anti-PARP antibody. SM denotes size marker. E denotes eluate. W denotes washing fraction, and F denotes flow-through fraction.

FIG. 6 shows induction and purification of ADPL2 protein. The identity of eluates was confirmed by Western blot analysis using anti-His antibody. E denotes eluate. W denotes washing fraction, and F denotes flow-through fraction. SM denotes size marker.

FIG. 7 shows induction and purification of ADPL3 protein. The identity of eluates was confirmed by Western blot analysis using anti-PARP antibody. SM denotes size marker. E denotes eluate. W denotes washing fraction, and F denotes flow-through fraction.

FIG. 8 shows ELISA of ADPNF protein. SLE (systemic lupus erythematosus); RA (rheumatoid arthritis); DMS (polymyositis/myositis); SSc (systemic sclerosis). 200 ng of purified recombinant ADPNF protein was used. Each serum of healthy individual and that of rheumatic patient was diluted (1:200) with solution containing 1% BSA. Positivity is defined when the OD value (of sera of patients) is higher than the mean value (of sera of healthy donors)+3 SD (standard deviation).

FIG. 9 shows ELISA of ADPCF2K. 200 ng of purified recombinant ADPCF2K protein was used. Positivity is defined when the OD value (of sera of patients) is higher than the mean value (of sera of healthy donors)+3 SD (standard deviation).

FIG. 10 shows ELISA of ADPL2 protein. 200 ng of purified recombinant ADPL2 protein was used. Positivity is defined when the OD value (of sera of patients) is higher than the mean value (of sera of healthy donors)+3 SD (standard deviation).

FIG. 11 shows ELISA of ADPL3 protein. 200 ng of purified recombinant ADPL3 protein was used. Positivity is defined when the OD value (of sera of patients) is higher than the mean value (of sera of healthy donors)+3 SD (standard deviation).

FIG. 12 shows ELISA of Sm protein. The positive reactivity is defined when the value is >25 Units/ml. ELISA was carried out according to the instruction manual provided by the manufacturer (BL Diagnostica, Hamburg, Germany).

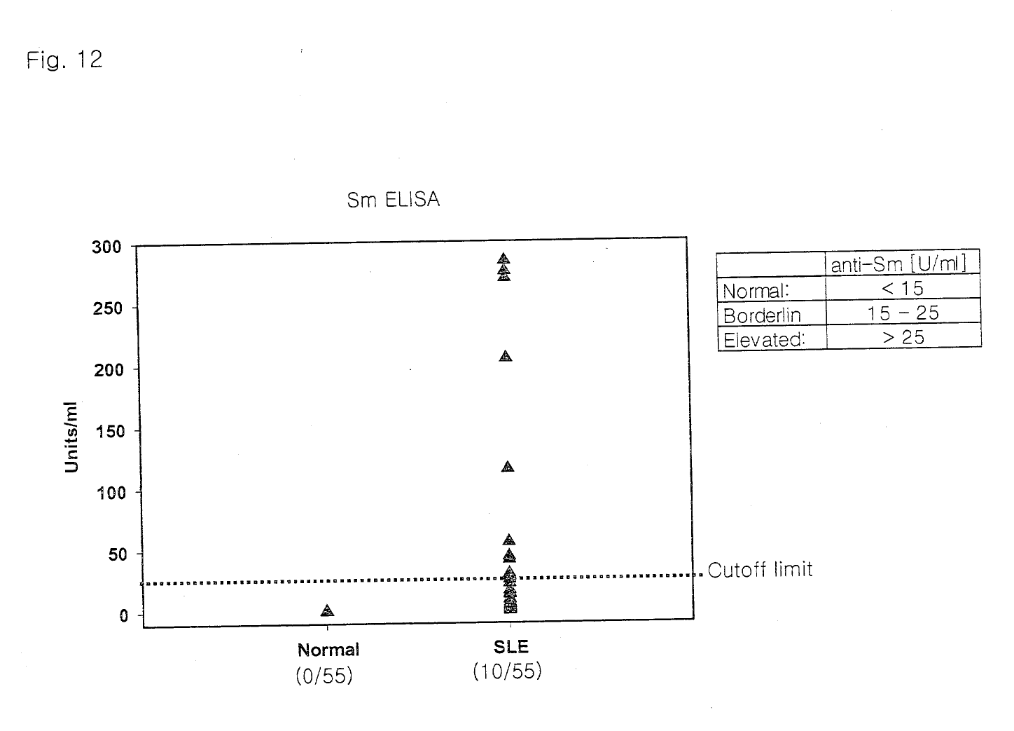

### DETAILED DESCRIPTION OF THE PREFERRED EMBODIMENTS

In the present application, “a” and “an” are used to refer to both single and a plurality of objects.

As used herein, “ADPCF2K” and “CF2K” are used interchangeably, as are “ADPNF” and “NF”; “ADPL2” and “L2”; “ADPL3” and “L3”.

As used herein, in general, the term “amino acid sequence variant” refers to molecules with some differences in their amino acid sequences as compared to a reference (e.g. native sequence) polypeptide. The amino acid alterations may be substitutions, insertions, deletions or any desired combinations of such changes in a native amino acid sequence.

Substitutional variants are those that have at least one amino acid residue in a native sequence removed and a different amino acid inserted in its place at the same position. The substitutions may be single, where only one amino acid in the molecule has been substituted, or they may be multiple, where two or more amino acids have been substituted in the same molecule.

Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threoninie, cysteine, tyrosine, asparagine and glutamine. The positively charged (basic) amino acids include arginine, lysine and histidine. The negatively charged (acidic) amino acids include aspartic acid and glutamic acid. Also included within the scope of the invention are proteins or fragments or derivatives thereof which exhibit the same or similar biological activity and derivatives which are differentially modified during or after translation, e.g., by glycosylation, proteolytic cleavage, linkage to an antibody molecule or other cellular ligand, and so on.

Insertional variants are those with one or more amino acids inserted immediately adjacent to an amino acid at a particular position in a native amino acid sequence. Immediately adjacent to an amino acid means connected to either the α-carboxy or α-amino functional group of the amino acid.

Deletional variants are those with one or more amino acids in the native amino acid sequence removed. Ordinarily, deletional variants will have one or two amino acids deleted in a particular region of the molecule.

As used herein, the term “antigen” is used in reference to any substance that is capable of reacting specifically with an antibody. It is intended that this term encompass any antigen and “immunogen” (i.e., a substance which induces the formation of antibodies). Thus, in an immunogenic reaction, antibodies are produced in response to the presence of antigen or portion of an antigen.

As used herein, the term “capable of hybridizing under high stringency conditions” means annealing a strand of DNA complementary to the DNA of interest under highly stringent conditions. Likewise, “capable of hybridizing under low stringency conditions” refers to annealing a strand of DNA complementary to the DNA of interest under low stringency conditions. “High stringency conditions” for the annealing process may involve, for example, high temperature and/or low salt content, which disfavor hydrogen-bonding contacts among mismatched base pairs. “Low stringency conditions” would involve lower temperature, and/or higher salt concentration than that of high stringency conditions. Such conditions allow for two DNA strands to anneal if substantial, though not near complete complementarity exists between the two strands, as is the case among DNA strands that code for the same protein but differ in sequence due to the degeneracy of the genetic code. Appropriate stringency conditions which promote DNA hybridization, for example, 6× SSC at about 45° C., followed by a wash of 2× SSC at 50° C. are known to those skilled in the art or can be found in CulTent Protocols in Molecular Biology, John Wiley & Sons, N.Y. (1989), 6.31-6.3.6. For example, the salt concentration in the wash step can be selected from a low stringency of about 2× SSC at 50° C. to a high stringency of about 0.2× SSC at 50° C. In addition, the temperature in the wash step can be increased from low stringency at room temperature, about 22° C., to high stringency conditions, at about 75° C. Other stringency parameters are described in Maniatis, T., et al., Molecular Cloning: A Laboratory Manual, Cold Spring Harbor Laboratory Press, Cold Spring N.Y., (1982), at pp. 387-389; see also Sambrook J. et al., Molecular Cloning: A Laboratory Manual, Second Edition, Volume 2, Cold Spring Harbor Laboratory Press, Cold Spring, N.Y. at pp. 8.46-8.47 (1989).

As used herein, “fragment” refers to a part of a polypeptide, which retains usable and functional characteristics. For example, as used within the context of the present invention, the polypeptide fragment has the function of binding to naturally occurring antibodies found in autoimmune patients.

As used herein, “His tag” refers to a molecular tag composed of amino acid histidine.

As used herein, “immunohistochemistry” refers to a method that measures level of specific protein in a variety of tissues.

As used herein, “immunoprecipitation” refers to a biological method that quantitatively measures expression level of a protein and also qualitatively the interaction between proteins.

As used herein, “ligand” refers to any molecule or agent, or compound that specifically binds covalently or transiently to a molecule such as a nucleic acid molecule or protein. Ligand may include antibody.

As used herein, “PARP” refers to poly ADP-ribose polymerase.

As used herein, “PARP polypeptide” refers to a polypeptide, which may be a fragment or derivative of PARP, which specifically binds to an antibody that is found naturally in a subject suffering from an autoimmune disease, such as but not limited to SLE. Preferably, the anti-PARP polypeptide antibody is statistically not found or found less naturally in normal human beings as compared with an autoimmune patient. While fragments of the PARP protein are included in the category of PARP polypeptides, the PARP polypeptide may have at least about 70%, 75%, 80%, 85%, 90%, 95%, 96%, 97%, 98%, 99% or 100% identity to the polypeptide sequence represented by SEQ ID NO:2, amino acid residues 300 to 1014, the C-terminal half of the PARP protein, or amino acid residues 681-1014.

Further, it is understood that various mutations and conservative amino acid changes are tolerable, as well as certain non-conservative amino acid changes, so long as the PARP polypeptide binds to the antibody that is naturally present in autoimmune patients. Fragments and certain glycosylations are also permitted, indeed any change at all to the PARP polypeptide is permitted so long as the polypeptide retains the ability to bind to naturally occurring antibody in autoimmune patients.

Applicants for the first time discovered that PARP polypeptide binds to naturally occurring antibodies from autoimmune patients, which allows the practitioner to diagnose patients suffering from autoimmune diseases, and thus it would be within the purview of a person of skill in the art to make certain variations to the sequence, which retains the capability of differentially binding such naturally occurring antibodies.

As used herein, “protein” refers to an amino acid sequence, polypeptide, oligopeptide, and polypeptide or portions thereof whether naturally occurring or synthetic.

As used herein, “purified” or “isolated” molecule refers to biological molecules that are removed from their natural environment and are isolated or separated and are free from other components with which they are naturally associated. As used herein, “RT-PCR” refers to a semi-quantitative PCR that uses cDNA as template rather than RNA.

As used herein, the term “reporter reagent” or “reporter molecule” is used in reference to compounds, which are capable of detecting the presence of antibody bound to antigen. For example, a reporter reagent may be a colorimetric substance, which is attached to an enzymatic substance. Upon binding of antibody and antigen, the enzyme acts on its substrate and causes the production of a color.

As used herein, “sample” or “biological sample” is referred to in its broadest sense. Any biological sample obtained from an individual, body fluid, cell line, tissue culture, or other source, which may contain anti-PART polypeptide antibodies, preferably anti-CF2K antibodies is included. As indicated, biological samples include body fluids, such as semen, lymph, sera, plasma, urine, synovial fluid, spinal fluid and so on. Methods for obtaining tissue biopsies and body fluids from mammals are well known in the art. Where the biological sample is to include mRNA, a tissue biopsy is the preferred source. As used herein, “SEREX” refers to Serological analysis of recombinant cDNA expression library.

As used herein, “sequence identity”, is defined as the percentage of amino acid residues in a candidate sequence that are identical with the amino acid residues in a native polypeptide sequence after aligning the sequences and introducing gaps, if necessary, to achieve the maximum percent sequence identity, and not considering any conservative substitutions as part of the sequence identity. The % sequence identity values are generated by the NCBI BLAST2.0 software as defined by Altschul et al., (1997), “Gapped BLAST and PSI-BLAST: a new generation of protein database search programs”, Nucleic Acids Res., 25:3389-3402. The parameters are set to default values, with the exception of the Penalty for mismatch, which is set to −1.

As used herein, the term “specifically binds” refers to a non-random binding reaction between two molecules, for example between an antibody molecule immunoreacting with an antigen, or a non-antibody ligand reacting with another polypeptide such as PARP.

As used herein, “subject” is a vertebrate, preferably a mammal, more preferably a human.

Identification of Autoantibodies in Sera of SLE Patients

To identify autoantibodies present in the sera of patients with SLE, we carried out SEREX (serological analysis of recombinant cDNA expression library). For this, cDNA expression libraries were constructed. These cDNA expression libraries were constructed from testis, kidney, and human lung cancer cell line NCIH1703. The titer of each of these cDNA expression libraries was on average 2×10⁶ pfu/ml. Each of these cDNA expression libraries was screened with pooled sera (1:250 dilution) of 10 patients with SLE. A total of 23 clones were found react with pooled sera of patients with SLE. After in vivo excision, identities of these clones were determined by sequencing.

There were 11 independent clones that reacted with pooled sera of 10 patients with SLE. Table 1 shows list of such genes. Table 1 shows list of genes identified by SEREX of cDNA expression libraries. NCI-H1703 is a lung cancer cell line, adenocarcinoma, squamous cell line.

TABLE 1

| Des- | | | | | |
|---|---|---|---|---|---|
| igna- | No. | Accession | Length | | |
| tion | Clone | No. | (Kb) | Gene | Source |
| S1 | 1 | XM012219 | 1.7 | Gene similar to | NCI- |
| | | | | phosphoglycerate mutase | H1703* |
| S2 | 2 | XM028790 | 2.9 | Homologous to Zfp161 | NCI- |
| | | | | in mouse | H1703 |
| S3 | 5 | NM001618 | 3.9 | Poly (ADP-ribose) | NCI- |
| | | | | polymerase | H1703 |
| S4 | 7 | BC001047 | 1.9 | Nucleolar autoantigen | NCI- |
| | | | | (55 KDa) | H1703 |
| S5 | 1 | NM004703 | 3 | Rabaptin-5 | NCI- |
| | | | | | H1703 |
| S6 | 1 | XM047728 | 1.9 | H2A histone family, | NCI- |
| | | | | member Y | H1703 |
| S7 | 1 | M95724 | 3.1 | Centromere autoantigen | NCI- |
| | | | | C | H1703 |
| S9 | 2 | M22636 | 1.7 | U1 small nuclear | NCI- |
| | | | | ribonucleoprotein | H1703 |
| | | | | 70 KDa protein | |
| S11 | 3 | NM002520 | 0.88 | nucleophosmin | testis |
| S14 | 1 | XM005953 | 5.7 | Transcription factor- | kidney |
| | | | | like 1 | |
| S15 | 1 | XM051637 | 0.9 | Galectin-3, galactose | kidney |
| | | | | binding | |

NCI-H1703*: lung cancer cell line, adenocarcinoma, squamous cell

All of these clones in Table 1 have significant homologies with known genes. Among these, nucleolar autoantigen (55 KDa), and PARP are represented in seven, and five overlapping clones, respectively. PARP is activated by DNA strand breaks.

Human PARP (113 KDa) is highly conserved and abundant protein. PARP is a DNA-dependent enzyme that catalyzes ADP-ribosylation of nuclear acceptor proteins by using NAD+ as a substrate (Althaus FR et al., Mol. Biol. Biochem. Biophys., 1987, 37: 1-126; de Murcia G et al., BioEssays, 1991, 13: 455-462; Shall S et al., Biochem. Soc. Trans. 1988, 17: 317-322). Data suggest that PARP is important for maintaining genomic integrity. The presence of autoantibody against PARP is interesting because this enzyme is involved in DNA repair and apoptosis (Lindah1 T et al., Trends Biochem. Sci., 1995, 20: 405-411; Utz PJ et al., J. Exp. Med. 185: 843-854). Previously, it was reported that autoantibody against PARP was present in the sera of patients with autoimmune rheumatic diseases and bowel diseases. U1 sn RNP (small nuclear ribonucleoprotein) identified in this screen is known to be associated with SLE. The clones identified in this study are involved in diverse cellular functions such as signaling, DNA repair, and transcription.

Galectin-3 is a galactose- and IgE-binding protein secreted by inflammatory macrophages. It plays a potential role in macrophage extracellular matrix interaction. Deregulated expression of galectin-3 is associated with hepatocellular carcinoma. Rabaptin-5 binds to GTP-ase and is involved in membrane docking or fusion. Nucleophosmin is a nucleolar phosphoprotein that is more abundant in tumor cells than in normal resting cells. It was shown to be a substrate of cyclin E/CDK2.

In this invention, the inventors did not detect autoantibody to ds DNA or Sm protein, which are well-known markers of SLE. This might be due to the method we used in this invention. Detection of autoantibodies by SEREX gives unbiased selection unlike Western blot or immunofluorescence staining. The inventors determined frequencies of IgG autoantibodies against the autoantigens. The inventors limited their analysis to IgG antibodies by using 1:250 diluted sera of patients with SLE for the screening of cDNA expression library and by using anti-human IgG antibody conjugated with alkaline phosphatase. To determine frequency of each autoantibody, λ-ZAP vector without insert was mixed with test clone. These coplated plaques were reacted with sera of patients with various rheumatic diseases including SLE and the detection of immunoreactivity was carried out by color reaction using NBT and BCIP.

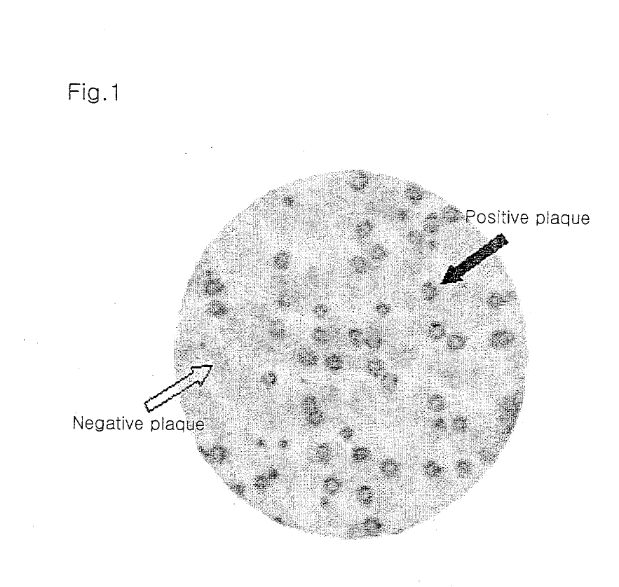

FIG. 1 shows reactivity of patient with SLE for PARP. Table 2 shows results of screening of allogenic sera for reactivity of autoantigens with IgG autoantibodies in the sera of patients with rheumatic diseases or healthy donors. SLE (systemic lupus erythematosus); RA (rheumatoid arthritis); DMS (polymyositis/myositis); SSc (systemic sclerosis).

TABLE 2

| Designation | SLE | RA | DMS | SSc | Sjogren | Normal |
|---|---|---|---|---|---|---|
| S1 | 3/68 | 0/50 | 0/37 | 1/18 | 0/19 | 0/76 |
| S2 | 5/68 | 0/50 | 0/37 | 0/18 | 0/19 | 0/76 |
| S3 | 26/68 | 1/50 | 0/37 | 0/18 | 0/19 | 0/76 |
| S4 | 2/68 | 3/50 | 1/37 | 0/18 | 0/19 | 1/76 |
| S5 | 1/68 | 0/50 | 0/37 | 0/18 | 0/19 | 0/76 |
| S6 | 4/68 | 0/50 | 0/37 | 0/15 | 0/19 | 0/76 |
| S7 | 5/68 | 0/50 | 0/37 | 12/18 | 0/19 | 0/76 |
| S9 | 25/68 | 0/50 | 2/37 | 3/18 | 0/19 | 0/76 |
| S11 | 4/57 | 0/50 | 0/37 | 1/18 | 0/19 | 1/56 |
| S14 | 14/68 | 1/50 | 0/37 | 0/18 | 0/19 | 0/56 |
| S15 | 12/63 | 0/50 | 16/37 | 0/18 | 0/19 | 0/56 |

The inventors found that PARP (26/68) and U1snRNP (25/68) showed the highest frequencies in sera of patients with SLE. U1snRNP was also present in the sera of patients with polymyositis/myositis (2/37) and systemic sclerosis (3/18). U1 snRNP was shown to interact with phosphorylated serine/arginine (SR) splicing factor in cells undergoing apoptosis. This indicates that SLE is related to regulation of alternative splicing of apoptotic effector molecules. Many of the autoantibodies in Table 2 showed specificity for sera of patients with SLE. For example, autoantibody to PARP did not react with sera of patients with Sjogren's syndrome or SSc or myositis while it showed low frequency in the sera of patients with RA (2%). This suggests that PARP is a valuable serologic marker for diagnosis of SLE. Most of the clones in Table 2 did not react with sera of healthy donors. This suggests that these autoantibodies are specific for autoimmune diseases. In this invention, we did not detect autoantibody against Sm protein, which is a well-known marker of SLE. This indicates racial variation in frequency of autoantibodies associated with SLE. The high prevalence of PARP in the sera of patients with SLE gives glimpse of the mechanism of pathogenesis leading to SLE. PARP, just like other targets for autoantibodies (U1snRNP, DNA topoisomeraes, and galectin-3), is cleaved during apoptosis. Serial analysis of sera taken at different stages during the course of disease is necessary for determining the value of these autoantibodies as markers for disease activity and the definition of clinically distinct subgroups of patients.

PARP Polypeptide

PARP (poly ADP-ribose polymerase) is a chromatin-bound, DNA-dependent enzyme that catalyzes the ADP-ribosylation of nuclear acceptor proteins by using NAD+as substrate. The resulting homopolymer, poly (ADP-ribose), has a branched structure containing phosphodiester and ribose bonds. Enzymes that are covalently modified by poly (ADP-ribosylation) are generally inhibited. This has been described for DNA topoisomerases I and II, DNA polymerases α and β, RNA polymerase II, ribonuclease, and for PARP itself. Poly (ADP-ribosylation) of nuclear proteins has thus been corroborated with a variety of regulatory functions such as DNA repair, gene expression, and cell differentiation. PARP also catalyzes the transfer of ADP ribose residues to monoenzyme acceptor proteins in the nucleus, such as histones H1 and H2, high mobility group proteins 1 and 2, or lamins, and this unique change represents the most drastic post-translational protein modification that can affect chromatin structure.

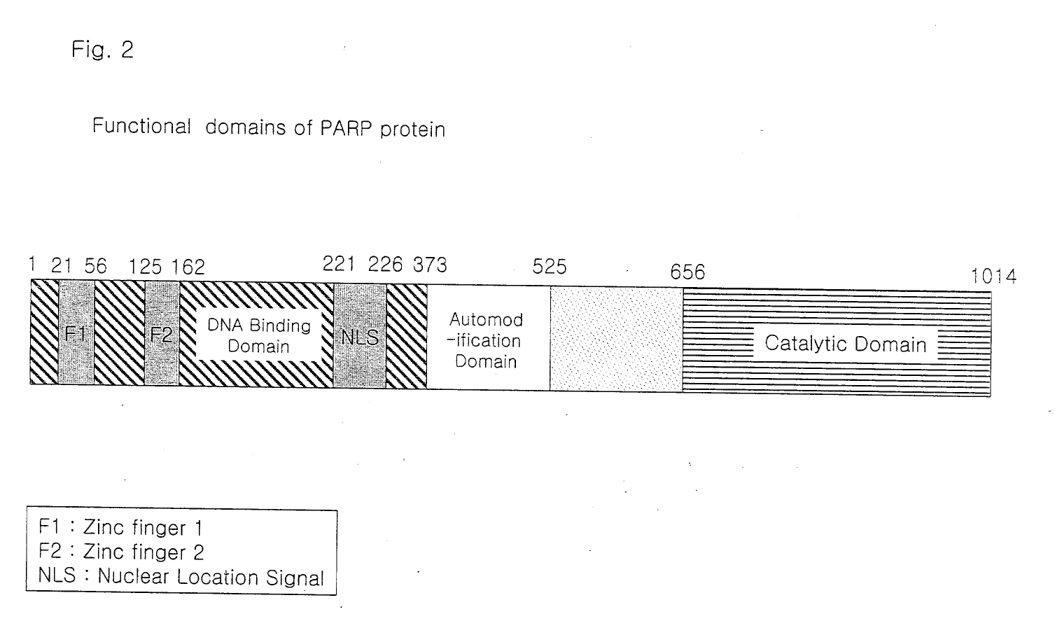

Human PARP contains 1014 amino acid residues (113 KD) and is a highly conserved and very abundant protein. From the analysis of natural fragments obtained by limited proteolysis of the purified enzyme, three functional domains have been identified (FIG. 2): i) a 46-KDa fragment including the DNA-binding domain (DBD), located in the N-terminal region; ii) a central hydrophilic 22 KDa polypeptide containing the automodification sites and a leucine zipper motif; and iii) a C-terminal fragment (54 KDa) bearing the NAD+domain which can be reduced to a 40 KDa fragment without losing its basal polymer synthesizing and branching activities (Simonin F et al., J. Biol. Chem. 1990, 265: 19249-19256). PARP is a zinc metalloenzyme (Zahradka P et al., Eur. J. Biochem., 1984, 142: 503-509; Mazen A et al., Nucleic Acids Res. 1989, 17:4689-4697), which contains two finger motifs, located in the DBD in residues 21-56 and 125-162 for finger 1 (F1) and finger 2 (F2) of human PARP, respectively. These two zinc fingers show homologous sequences probably resulting from internal gene duplication and have no sequence homology to other existing zinc-finger classes. F1 and F2 are involved in the specific recognition of single and double strand breaks in DNA. The DBD also contains nuclear location signal for nuclear homing of PARP. The inventors constructed expression vectors containing various domains of PARP protein.

The inventors constructed four different expression vectors containing various functional domains of PARP protein. ADPNF contains amino acid residues 1-234 of PARP. ADPNF contains two zinc finger domains of PARP. ADPCF2K contains amino acid residues 300-1014. It contains automodification domain and catalytic domain (NAD+-binding domain). ADPL3 contains amino acid residues 681-1014. It contains catalytic domain (NAD+-binding domain). ADPL2 contains amino acid residues 339-680. It contains automodification domain. Functional domains of PARP were cloned into pET2 la vector.

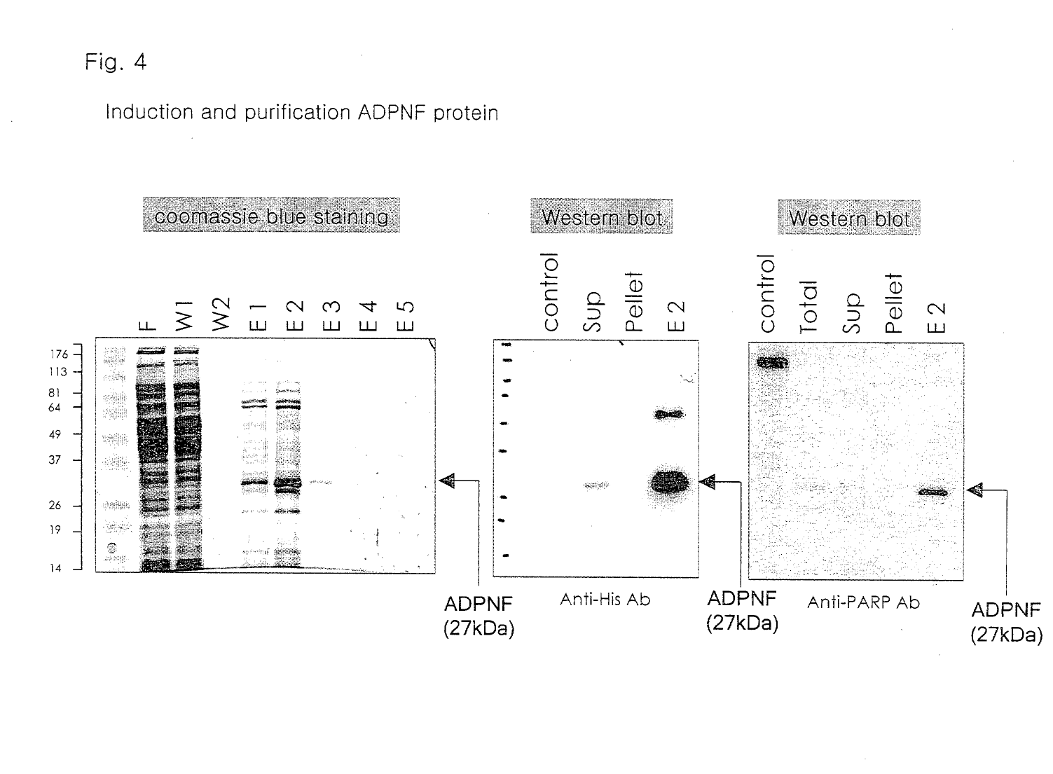

Thus obtained expression vectors contain His tag. Each of these expression vectors was transformed into E. coli. Crude lysates of IPTG-induced bacteria, corresponding to 10 Vtl of bacterial culture, were subjected to 10% SDS-PAGE followed by Western blot analysis. FIG. 4 shows induction of ADPNF protein. In this, 0.1 mM of IPTG was used for the induction of ADPNF protein. The purification of ADPNF protein was carried out by affinity chromatography using Ni²⁺-resin according to the instruction manual provided by the manufacturer (Qiagen Company, Westburg, Leusden, Netherlands). Western blot analysis using either anti-His Ab or anti-PARP Ab was carried out to confirm identity of ADPNF protein.

PARP Polypeptide Reactivity with Autoantibody

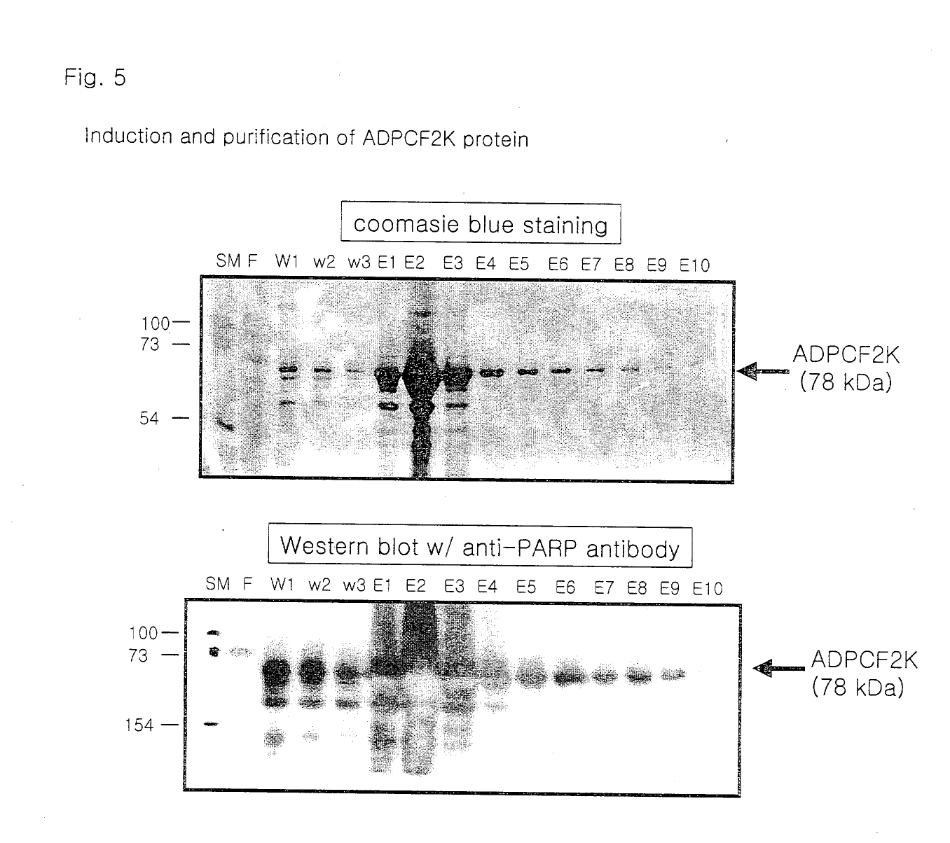

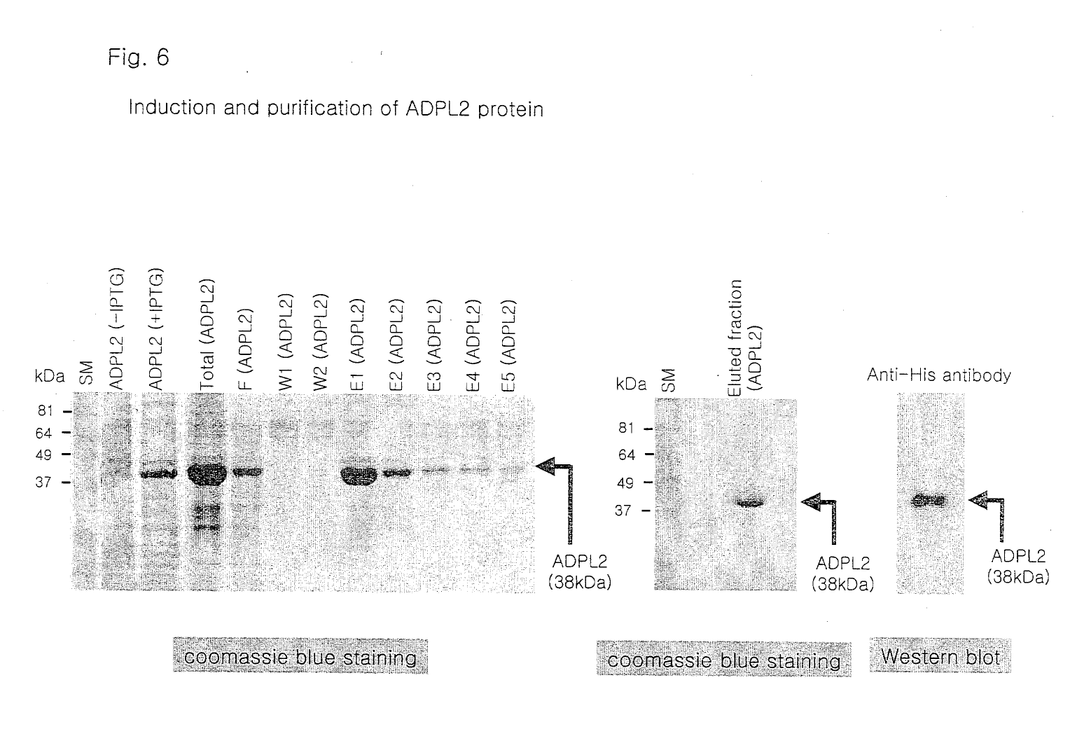

FIG. 5 shows induction of ADPCF2K protein. In this, 0.1 mM of IPTG was used for the induction of ADPCF2K protein. The purification of ADPCF21K protein was carried out by affinity chromatography using Ni²⁺-resin according to the instruction manual provided by the manufacturer (Qiagen Company, Westburg, Leusden, Netherlands). Western blot analysis using anti-PARP Ab was carried out to confirm identity of ADPCF2K protein. Elution fractions E4-E7 were collected and used for ELISA for the diagnosis of rheumatic diseases. FIG. 6 shows induction of ADPL2 protein. In this, 0.2 mM of IPTG was used for the induction of ADPL2 protein. The purification of ADPL2 protein was carried out by affinity chromatography using Ni²⁺-resin according to the instruction manual provided by the manufacturer (Qiagen Company, Westburg, Leusden, Netherlands). Western blot analysis using anti-PARP Ab was carried out to confirm identity of ADPL2 protein (data not shown).

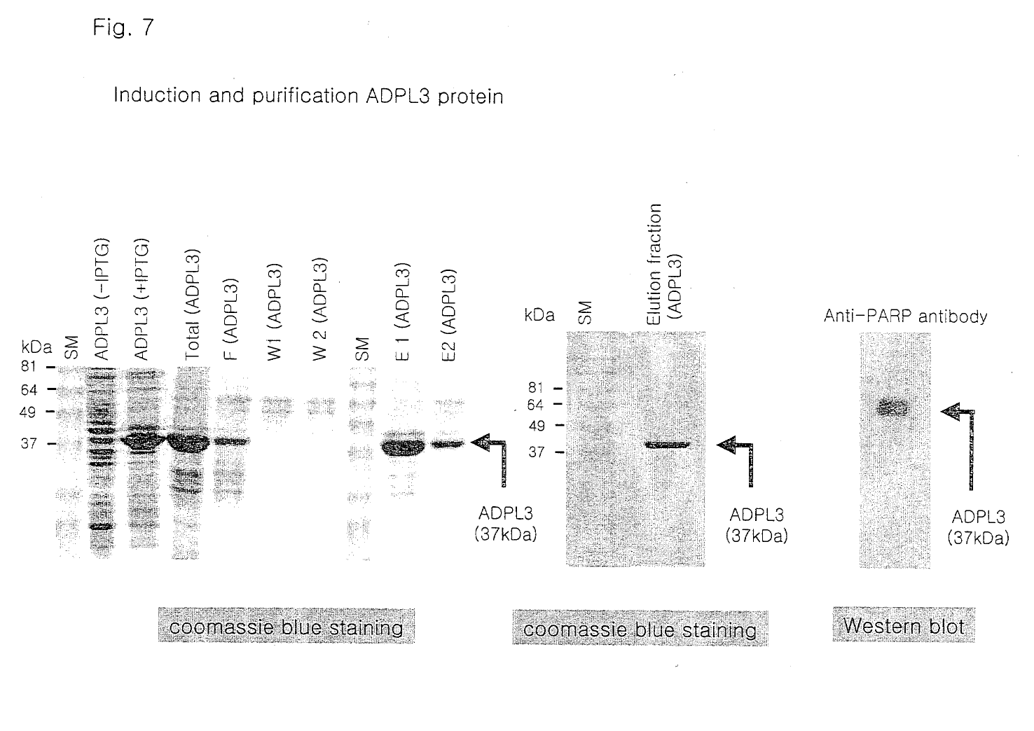

FIG. 7 shows induction of ADPL3 protein. In this, 0.2 mM of IPTG was used for the induction of ADPL3 protein. The purification of ADPL3 protein was carried out by affinity chromatography using Ni²⁺-resin according to the instruction manual provided by the manufacturer (Qiagen Company, Westburg, Leusden, Netherlands). Western blot analysis using anti-PARP Ab was carried out to confirm identity of ADPL3 protein (data not shown).

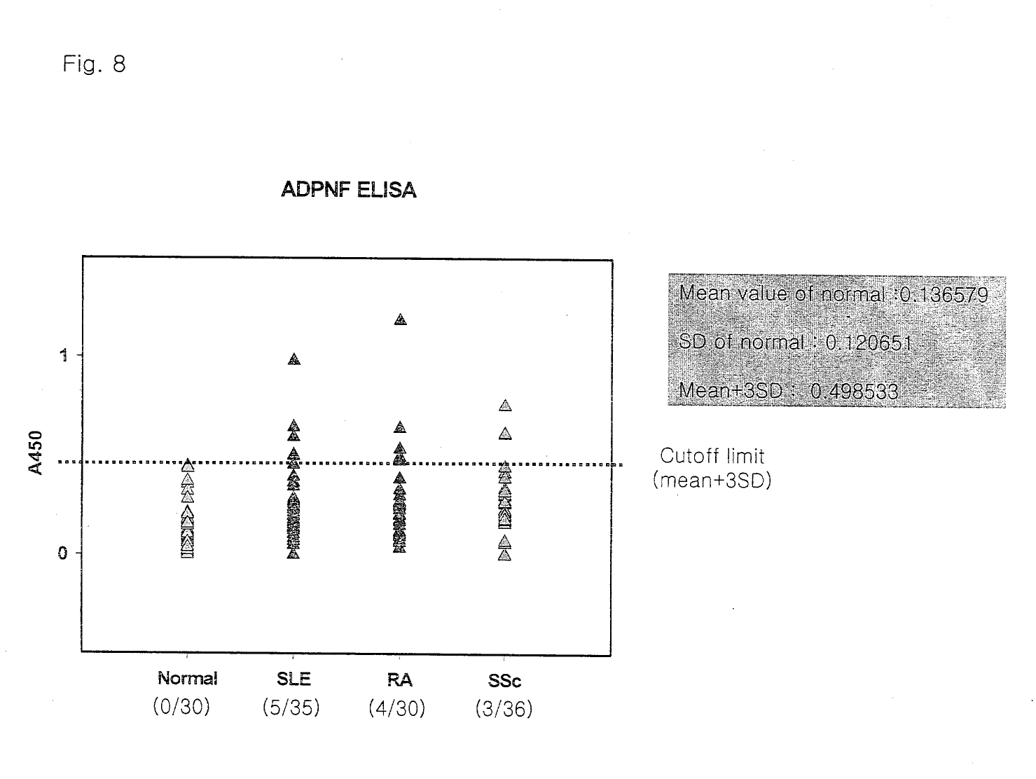

FIG. 8 shows ELISA results of ADPNF. In this, the cutoff points were determined with a series of sera from healthy individuals. Sera were considered positive when the OD values were higher than the mean OD value of these normal sera+3SD. In this assay, 14% (5/35) of sera of patients with SLE showed positive value. Sera from patients with RA (rheumatoid arthritis) showed moderate reactivity (4/30). Sera from patients with systemic sclerosis showed weak reactivity (3/36). None out of thirty healthy individuals showed positive value.

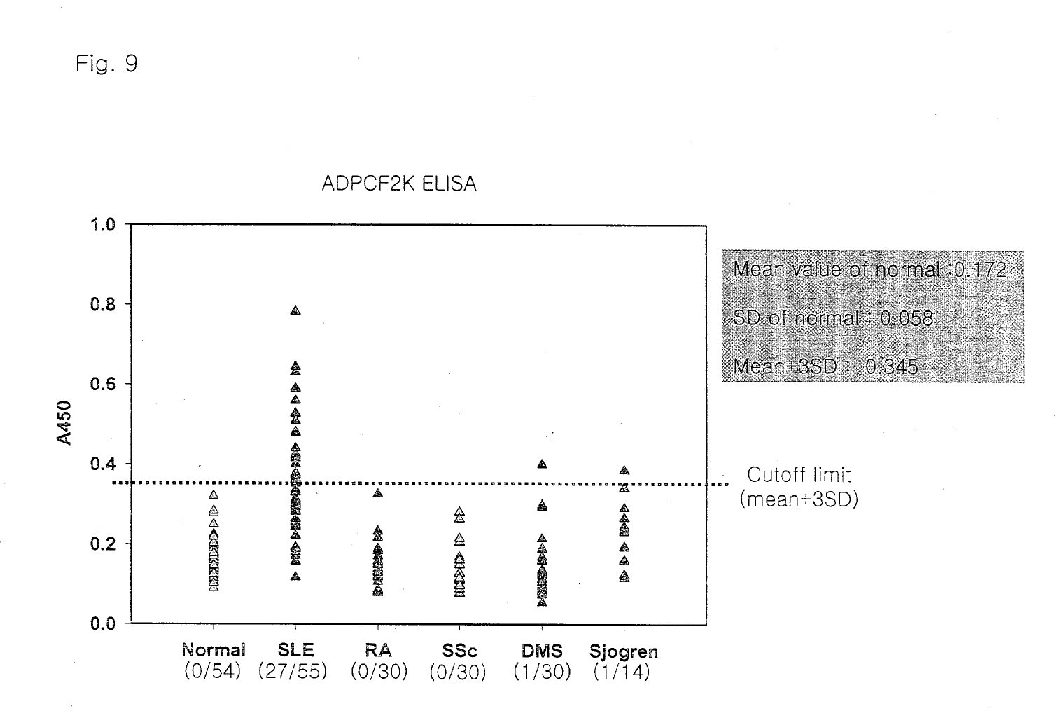

FIG. 9 shows ELISA results of ADPCF2K using sera of various rheumatic patients or those of healthy donors. In this, the cutoff points were determined with a series of sera from healthy individuals. Sera were considered positive when the OD values were higher than the mean OD value of these normal sera+3SD. In this assay, 49% (27/55) of sera of patients with SLE showed positive value. Sera from patients with RA (rheumatoid arthritis) and SSc (systemic sclerosis) showed no reactivity (0/30). Sera from DMS (polymyositis/myositis) showed weak reactivity (1/30). Sera from patients with Sjogren syndrome showed weak reactivity (1/14).

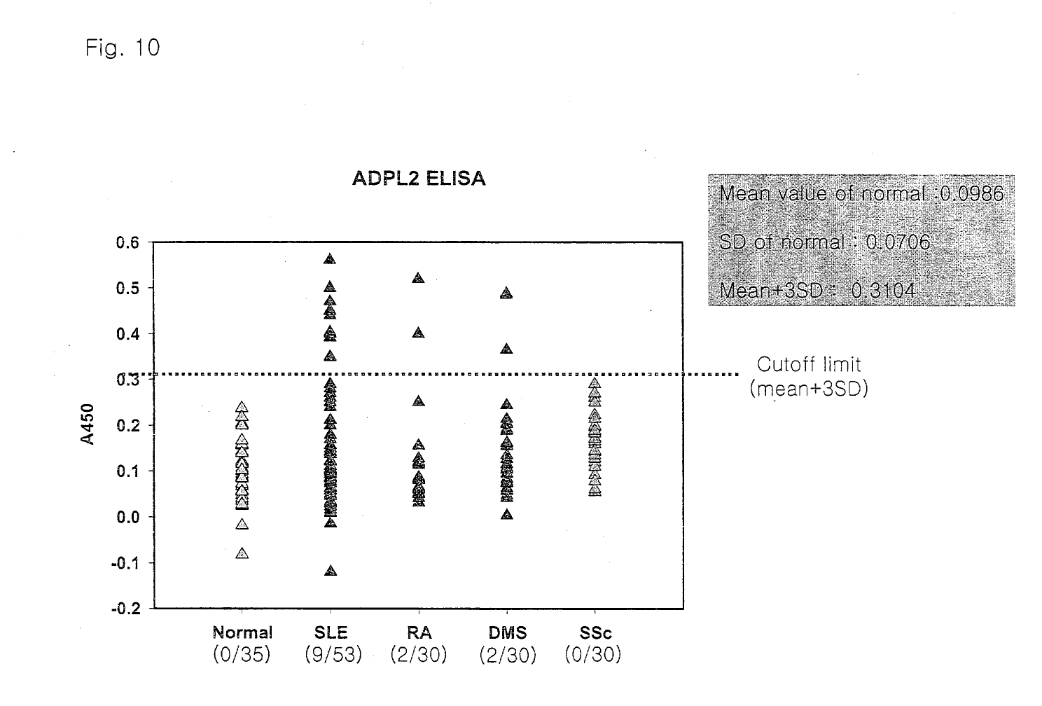

FIG. 10 shows ELISA results of ADPL2 using sera of various rheumatic patients or those of healthy donors. In this, the cutoff points were determined with a series of sera from healthy individuals. Sera were considered positive when the OD values were higher than the mean OD value of these normal sera+3SD. In this assay, 17% (9/53) of sera of patients with SLE showed positive value. Sera from patients with RA (rheumatoid arthritis) showed weak reactivity (2/30). Sera from DMS (polymyositis/myositis) also showed weak reactivity (2/30). Sera from patients with systemic sclerosis showed no reactivity (0/30).

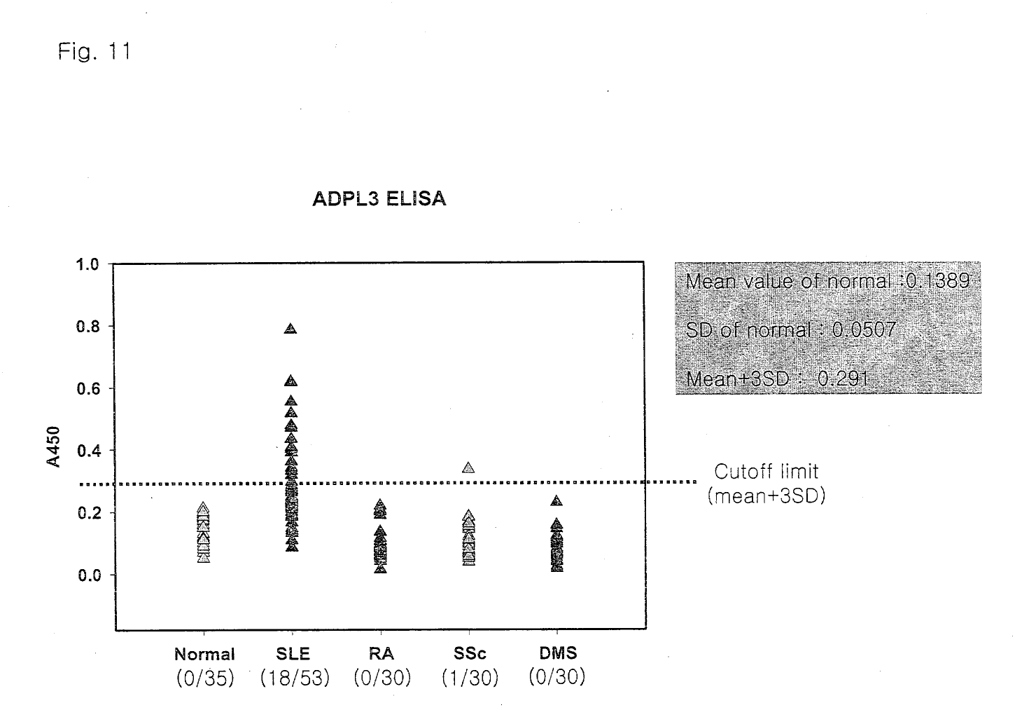

FIG. 11 shows ELISA results of ADPL3 using sera of various rheumatic patients or those of healthy donors. In this, the cutoff points were determined with a series of sera from healthy individuals. Sera were considered positive when the OD values were higher than the mean OD value of these normal sera+3SD. In this assay, 34% (18/53) of sera of patients with SLE showed positive value. Sera from patients with RA (rheumatoid arthritis) showed no reactivity (0/30). Sera from DMS (polymyositis/myositis) showed no reactivity (0/30). Sera from patients with systemic sclerosis showed no reactivity (1/30). High sensitivity and specificity of ELISA result using ADPL3 protein suggests that ADPL3 protein would be valuable marker for the diagnosis of SLE. These ELISA results suggest that domain of PARP protein would be a valuable serologic marker for the diagnosis of SLE.

Table 3 shows clinical characteristics of patients with SLE. The presence of autoantibodies against PARP is inversely related with Pleurisy. Chi-square test was used in statistical analysis.

TABLE 3

| | Positive (n = 26) | Negative (n = 42) | P-value |
|---|---|---|---|
| Age (yrs, mean ± SD) | 30.7 ± 9.8 | 37.3 ± 12.2 | 0.02 |
| Disease duration | 58.2 ± 48.7 | 68.3 ± 44.3 | 0.38 |
| (months) | | | |
| Malar rash | 15/26 | 21/42 | 0.54 |
| Discoid lesion | 1/26 | 1/42 | 1.0 |
| Oral ulcer | 10/26 | 16/42 | 0.98 |
| Photosensitivity | 9/26 | 8/42 | 0.15 |
| Arthritis | 18/26 | 29/42 | 0.99 |
| Pleurisy | 3/26 | 17/42 | 0.01 |
| Percarditis | 1/26 | 8/42 | 0.13 |
| Hemolytic anemia | 2/26 | 11/42 | 0.11 |
| Leukepenia | 15/26 | 25/42 | 0.88 |
| Thrombocytopenia | 3/26 | 13/42 | 0.06 |
| Renal disease | 14/26 | 18/42 | 0.38 |
| CNS disorder | 1/25 | 7/42 | 0.14 |
| Anti-ds DNA Ab | 24/26 | 36/40 | 0.75 |
| Ant-Sm Ab | 4/10 | 4/15 | 0.48 |

Table 4 shows summary of ELISA results using various recombinant proteins. SLE (systemic lupus erythematosus); RA (rheumatoid arthiritis); DMS (polymyositis/myositis); SSc (systemic sclerosis). The presence of autoantibodies to CF2K was analyzed to determine if it was associated with the clinical subset of SLE.

TABLE 4

| | | | | | | | p-value |
|---|---|---|---|---|---|---|---|
| | | | | | | Nor- | (Chi- |
| Antigen | SLE | RA | DMS | SSc | Sjogren | mal | square) |
| ADP- | 27/55 | 0/30 | 1/30 | 0/30 | 1/14 | 0/54 | |
| CF | (49.1) | (0) | (3.3) | (0) | (7.1) | (0) | 0.00053 |
| ET-L2 | 9/53 | 2/30 | 2/30 | 0/30 | nd | 0/35 | |
| | (17) | (6.7) | (6.7) | (0) | | (0) | 0.00396 |
| ET-L3 | 18/53 | 0/30 | 0/30 | 1/30 | nd | 0/35 | |
| | (34) | (0) | (0) | (3.3) | | (0) | 5.8E−06 |
| ADP- | 5/35 | 4/30 | nd | 3/36 | nd | 0/30 | |
| NF | (14.3) | (13.3) | | (8) | | (0) | 0.00852 |

Cutoff point: Mean value of normal sera + 3SD
nd: not determined

Further, shown in Table 5, the presence of autoantibodies to CF2K did not show correlation with any of clinical subsets of SLE. Table 5 shows clinical and serological characteristics of SLE patients with or without anti-CF2K antibodies. The clinopathological characteristics of 49 SLE patients with or without anti CF2K antibodies were subjected to analysis.

TABLE 5

| | P-value (Chi-square) |
|---|---|
| Sex | 0.81 |
| Malar rash | 0.7 |
| Discoid lesion | 0.39 |
| Oral ulcer | 0.24 |
| Photosensitivity | 0.16 |
| Arthritis | 0.84 |
| Pleurisy | 0.36 |
| Pericarditis | 0.79 |
| Hemolytic anemia | 0.5 |
| Leukepenia | 0.9 |
| Thrombocytopenia | 0.5 |
| Nephritis | 0.63 |
| CNS disorder | 0.3 |
| ANA | 0.76 |
| Anti-ds DNA Ab | 0.069 |
| VDRL | 0.47 |
| LE cell | 0.91 |
| Age | 0.49 |
| Duration | 0.64 |
| anti-DNA titer | 0.73 |

The presence of autoantibodies to CF2K did not show correlation with the presence of anti-dsDNA or ANA (antinuclear antibody) indicating that the autoantibodies to CF2K would be a serologic marker, distinct from autoantibodies to dsDNA, for the detection of SLE.

It is known that many autoantigens are positioned in a close spatial relationship. For example, the Sm and the various ribonucleoproteins RNP form a ribonucleoprotein complex, which is present in the core of eukaryotic cells (snRNP). Anti-sm antibodies recognize various proteins of the snRNP complex, which are designated as B′, B, D, E, F, and G. This complex probably plays a central role in the splicing of the pre-mRNA, wherein the Sm-D protein represents an important regulation protein, in that it is to regulate the binding of various ribonucleic acids. It is undetermined which mechanisms lead to the formation of autoantibodies. It is noticeable that particularly important regulatory centers for the cell cycle are the goal of autoantibodies of the SLE and of syndromes related to SLE. Bloom and coworkers proved in connection with investigations of hybridoma cell lines, produced by means of hybridoma techniques, that anti-Sm antibodies could also recognize DNA (D. D. Bloom et al., Journal of Immunol. 1993; 150(4): 1579-1590). The authors conclude that the Sm-D protein by itself does not act as antigen for the formation of anti-sm antibodies. Rather, it is assumed that both the Sm-D autoantigen and DNA, possibly as a complex, from the autoimmune agent. The physiological and pathophysiological importance of the Sm/DNA complex is not known up to now.

In addition to the antibodies against native or double-stranded DNA, anti-Sm antibodies are deemed to be a diagnostic marker for the systemic lupus erythematosus. A pathogenic role is attributed to the anti-Sm antibodies in the generation of damages to organs. The proof of the anti-Sm antibodies succeeds in Europe, contrary to the anti-ds DNA antibodies, only in the case of relatively few patients, whereas in the United States it can be determined in one third of the patients with SLE. The cause of this is considered to be a different ethnic composition of the population (N. Abuaf et al., Eur. J. Clin. Invest. 1990; 20: 354-359). In this invention, we checked whether Sm protein was an appropriate marker for the detection of SLE. For this, we used commercially available kit containing Sm-D protein. We carried out ELISA of Sm protein using commercially available kit. For this, 55 sera of patients with SLE and the same number of sera of healthy donors were subjected to ELISA. ELISA was carried out according to the instruction manual provided by the manufacturer (BL Diagnostica, Hamburg, Germany). In this assay, 18% (10/55) of sera of patients with SLE showed positive value. According to the instruction manual, positive reactivity is defined when the value is >25 Units/ml.

In one aspect of the invention, throughout ELISA experiments, a fragment of PARP protein, and in particular, ADPCF2K and ADPL3, showed high sensitivity and specificity for the diagnosis of SLE. This PARP polypeptide showed even higher sensitivity than Sm protein, which is being currently used for the diagnosis of SLE.

Diagnostic Assay

The invention also provides diagnostic methods for detecting the presence of anti-PARP polypeptide antibodies in a biological sample. This may be assayed either directly or indirectly.

Where diagnosis of a diseased state has already been made, the present invention is useful for monitoring progression or regression of the disease state by measuring the amount of PARP polypeptide/anti-PARP polypeptide antibody complex present in an autoimmune patient or whereby patients exhibiting enhanced anti-PARP polypeptide antibody production will experience a worse clinical outcome relative to patients producing anti-PARP polypeptide antibody at a lower level.

Presence of the labeled molecule can be detected in the patient using methods known in the art for in vivo scanning. These methods depend upon the type of label used. Skilled artisans will be able to determine the appropriate method for detecting a particular label. Methods and devices that may be used in the diagnostic methods of the invention include, but are not limited to, computed tomography (CT), whole body scan such as position emission tomography (PET), magnetic resonance imaging (MRI), and sonography, as well as, electron paramagnetic resonance (EPR) and nuclear magnetic resonance (NMR).

In a specific embodiment, a PARP polypeptide is labeled with a radioisotope and is detected in the patient using a radiation responsive surgical instrument (Thurston et al., U.S. Pat. No. 5,441,050). In another embodiment, the molecule is labeled with a fluorescent compound and is detected in the patient using a fluorescence responsive scanning instrument. In another embodiment, the molecule is labeled with a positron emitting metal and is detected in the patient using positron emission-tomography. In yet another embodiment, the molecule is labeled with a paramagnetic label and is detected in a patient using magnetic resonance imaging (MRI).

The present invention is not to be limited in scope by the specific embodiments described herein. Indeed, various modifications of the invention in addition to those described herein will become apparent to theose skilled in the art from the foregoing description and accompanying figures. Such modifications are intended to fall within the scope of the appended claims. The following examples are offered by way of illustration of the present invention, and not by way of limitation.

### EXAMPLES

### Example 1

### Patients

Sixty eight Patients with SLE were enrolled at the Rheumatology Clinic, Department of Medicine at Seoul National University. The patients' age was 34.7±11.7 years old (means±SD) with 96% being female. The disease duration was 64.4±45.9 months.

### Example 2

### Construction of cDNA Expression Library

A total of 5 μg of human testicular mRNA (Clontech Company, Palo Alto, Calif., USA) or mRNA of NCIH1703 or kidney tissue was used for the construction of cDNA expression library. Construction of each cDNA expression library was carried out according to the instruction manual provided by the manufacturer (Stratagene Company, La Jolla, Calif., USA). Briefly, messenger RNA was converted into cDNA by MMLV (Moloney Murine Leukemia Virus) reverse transcriptase. This single stranded cDNA, which contains Xho 1 restriction site, was converted into double stranded cDNA by DNA polymerase. To this double stranded cDNA, EcoRI adaptor sequence was added by T4 DNA ligase, then cut with XhoI to yield unidirectional cDNA, and subsequently ligated to λ ZAP express vector. Thus ligated cDNA was packaged into phage particles by using Gigapack III Gold packaging extract (Stratagene Company, La Jolla, Calif., USA), which was used to infect XL1-Blue MRF, an E. coli strain. The library consisted on average 2×10⁶ primary recombinants and 5×10⁶ of them were used for immunoscreening.

### Example 3

### Screening of cDNA Expression Library with Pooled Sera of Patients with SLE

Primary cDNA expression library (human testis or lung cancer or kidney cDNA expression library) was screened with pooled sera of patients with SLE. Screening procedure was done according to the instruction manual provided by the manufacturer (Stratagene Company). Briefly, pooled sera of patients with SLE were diluted 1:10 in blocking buffer (KPL), preadsorbed with transfected E. coli lysates, and incubated overnight at room temperature with the nitrocellulose membranes containing the phage plaques (104 plaques/100 mm dish). After washing, followed by incubation with secondary antibody, an anti-human IgG antibody was conjugated with alkaline phosphatase. The reactive phage plaques were visualized by incubation with NBT (Nitro Blue Tetrazolium, 0.3 mg/ml) and BCIP (5-bromo-4-chloro-3-indolyl-phosphate, 0.15 mg/ml). Immunoreactive clones were tested for reactivity toward diluted sera (1:250) of patients with SLE or those of normal healthy individuals by using same screening strategy. Sera of patients with SLE were provided by Prof. Y. Song (Seoul National University Hospital, Seoul, South Korea).

### Example 4

### Genes Isolated by SEREX of cDNA Expression Libraries

Immune-reactive cloned inserts were in vivo excised into the plasmid form according to the instruction manual provided by the manufacturer (Stratagene). Plasmid DNA was purified by commercial kit (Qiagen Company, Westburg, Leusden, the Netherlands). Sequencing reactions were performed by ABI PRISM 310 Genetic Analyzer automated sequencer (Perkin Elmer, Foster City, Calif., USA). Sequence homology searches were performed in the databases provided by the National Center for Biotechnology Information (Bethesda, Md., USA). We identified a total of 11 independent clones that reacted with pooled sera of patients with SLE. Most of these clones showed homology with known genes. Table 1 is a list of genes identified in this screen. The most frequently isolated genes were Nucleolar autoantigen (7/25), PARP (5/25), and nucleophosmin (3/25).

### Example 5

### Sensitivity and Specificity of PARP

Next, we determined frequencies of IgG autoantibodies against the autoantigens detected in this invention. The inventors limited their analyses to IgG antibodies by using 1:250 diluted sera of patients with SLE for the screening of cDNA expression library and by using anti-human IgG antibody conjugated with alkaline phosphatase. To determine frequency of each autoantibody, λ-ZAP vector without insert was mixed with test clone. These coplated plaques were reacted with sera of patients with various rheumatic diseases including SLE and the detection of immunoreactivity was carried out by color reaction using NBT (Nitro blue teatrazolium) and BCIP (5-Bromo-4-chloro-3-Indolyl-phosphate).

### Example 6

### Construction of Expression Vectors

The cDNA sequence of CF2K (SEQ ID NO:1) was amplified by PCR. PCR was performed for 25 cycles at 94° C. for 1 min, 60° C. for 1 min, and 72° C. for 2 min. Primers used are: 5′-AAG TC GAC GCT GGT CTT CAA GAG-3′ (forward, SEQ ID NO:3) and 5′-AAC TCG AGC CAC CGG GAG GTC T-3′ (reverse, SEQ ID NO:4). Subsequently obtained PCR product was cut with Sal I and Xho I, and subcloned into pET21 vector (Promnega). pET21 vector construct was cut with Xho I and Sal I. Ligation reaction was followed. Before ligation reaction, calf intestine alkaline phosphatase was added at 37° C. for 20 min. For cloning of L2, cDNA sequences of L2 were amplified by PCR. PCR was performed for 25 cycles at 94° C. for 1 min, 60° C. for 1 min, and 72° C. for 1 min. Primers used are: 5′-AAA GAA TTC TTC CGA GAA ATC TCT TAC C-3′ (forward, SEQ ID NO:5) and 5′-AAA CTC GAG TTC CAC ATC AAA GAT CAT C-3′ (reverse, SEQ ID NO:6). Subsequently obtained PCR products were cut with EcoR1 and Xho I and ligated with EcoR1/Xho1-digested pET21a vector (Novagen, Madison, Wis., USA). For cloning of L3, cDNA sequences of L3 were amplified by PCR. PCR was performed for 25 cycles at 94° C. for 1 min, 60° C. for 1 min, and 72° C. for 1 min. Primers used are: 5′-AAA GAA TTC AGT ATG AAG AAA GCC ATG G-3′ (forward, SEQ ID NO:7) and 5′-AAA CTC GAG CCA CAG GGA GGT CTT AA-3′ (reverse, SEQ ID NO:8). Subsequently obtained PCR product was cut with EcoR1 and Xho1 and ligated with EcoR1/Xho1-digested pGEX5X-1 vector (Amersham Pharmacia Biotech, Piscataway, N.J.). For cloning of NF, cDNA sequences of NF were amplified by PCR. PCR was performed for 25 cycles at 94° C. for 1 min, 55° C. for 1 min and 72° C. for 1 min. Primers used are: 5′-TCA AGC TTA CTA TCC TTG TC-3′ (forward, SEQ ID NO:9) and 5′-TCA AGC TTA CTA TCC TTG TC-3′ (reverse, SEQ ID NO:10). Subsequently obtained PCR product was cut with BamH1 and HindIII and ligated with BamH1/HindIIIdigested pGEX5X-1 vector (Novagen, Madison, Wis., USA).

### Example 7

### Transformation of expression vectors and Western blot analysis

PET21a-NF, pET21a-L2, pET21b-CF2K or pET21b-L3 construct was mixed with E. coli BL21 (DE3) and incubation continued on ice for 30 min. The mixture was heat shocked at 42° C. for 1 min. After heat shock, the strain was mixed with LB medium and incubation continued at 37° C. for 1 h. After reaction, E. coli was plated out onto LB/amp medium. After incubation at 37° C. for 14 h, colonies were picked, and grown for isolation of recombinant protein.

### Example 8

### Induction and Purification of Various Domains of PARP Protein

ADPNF and ADP-L2 protein in this invention was purified according to the standard procedures well known to the field. Briefly, the transformed E. coli BL21(DE3) was inoculated (1/100) into 200 ml LB/Amp medium and incubation continued at 37° C. for 3 h. After incubation, 0.1 mM IPTG was added and incubation continued at 37° C. for 3 h. E. coli lysates containing expression vectors were subjected to sonication. For purification of ADPNF and ADPL2 proteins, affinity column chromatography was carried out according to the standard procedures. Briefly, cultured cells with pET21a-ADPNF or pET21a-L2 construct treated with 0.11 mM IPTG was dissolved in PBS and sonicated for 5 min. Cell lysates were centrifuged at 15,000 rpm for 30 min. ADPNF and ADPL2 proteins went into pellet (inclusion body) and the pellet was dissolved in lysis buffer (1× PBS buffer, 8M urea pH8.0). Purification of ADPNF and ADPL2 proteins was carried out according to the instruction manual provided by the manufacturer using Ni²⁺-NTA agarose (Qiagen Company, Westburg, Leusden, Netherlands). Briefly, dissolved pellet was incubated with Ni²⁺-NTA agarose (0.5 ml for 200 ml E. coli culture) for 2 h and the lysate-resin mixture was loaded onto empty column. After washing twice with wash solution (1×PBS, 10 mM imidazole, 8M urea, pH8.0), ADPNF or ADPL2 protein was eluted with elution solution (1× PBS, 20 mM imidazole, 8M urea, pH8.0). Subsequently obtained recombinant protein was loaded onto 12% SDS-PAGE. After electrophoresis, the gel was incubated with 100 mM KCl and the gel piece containing protein of interest was homogenized. The incubation was continued at 4° C. for 16 h. After incubation, each protein was centrifuged at 10,000 rpm for 20 min and the supernatant was saved for further use. The supernatant was loaded for 12% SDS-PAGE to check the purity.

CF2K protein (SEQ ID NO:2) in this invention were purified according to the standard procedures well known to the field. Briefly, the transformed E. coli BL21 (DE3) was inoculated (1/100) into 200 ml LB/Amp medium and incubation continued at 37° C. for 3 h. After incubation, 0.1 mM IPTG was added and incubation continued at 37° C. for 3 h. E. coli lysates containing expression vectors were subjected to sonication. For purification of CF2K protein, affinity column chromatography was carried out according to the standard procedures. Briefly, cultured cells with pET21b-CF2K construct treated with 0.1 mM IPTG was dissolved in PBS and sonicated for 5 min. Cell lysates were centrifuged at 15,000 rpm for 30 min. The supernatant was saved for affinity column chromatography using Ni²⁺-NTA agarose (Qiagen Company, Westburg, Leusden, Netherlands). Briefly, the supernatant was incubated with Ni²⁺-NTA agarose (0.5 ml for 200 ml E. coli culture) for 2 h at 4° C. and the lysate-resin mixture was loaded onto empty column. After washing twice with wash solution (1× PBS, 20 mM imidazole, pH7.2), CF2K protein was eluted with elution solution (1× PBS, 200 mM imidazole, pH7.2).

For Western blot analysis, each elution fraction was run on 10% SDS-PAGE. After electrophoresis, proteins were transferred onto PVDF membrane at 4° C. for 2 h at 200 mA. Membrane was incubated with blocking buffer for 1 h. After blocking, the membrane was incubated with monoclonal anti-PARP Ab (1:5,000 dilution) or polyclonal anti-1H is Ab (1:3,000 dilution) for 1 h. Membrane was washed with buffer (1× PBS, 0.2% Tween 20 (vol/vol)) for a total of 30 min. After washing, membrane was incubated with HRP-conjugated anti mouse Ab (1:15,000 dilution) or HRP-conjugated anti rabbit Ab (1:15,000 dilution) for 1 h. After washing, detection of protein of choice was carried out by using ECL kit according to the manufacturer's protocol (Amnersham International, England).

ADPL3 protein in this invention was purified according to the standard procedures well known to the field. Briefly, the transformed E. coli BL21 (DE3) was inoculated (1/100) into 200 ml LB/Amp medium and incubation continued at 37° C. for 3 h. After incubation, 0.1 mM IPTG was added and incubation continued at 37° C. for 3 h. E. Coli lysates containing expression vectors were subjected to sonication. For purification of ADPL3 protein, affinity column chromatography was carried out according to the standard procedures. Briefly, cultured cells with pET21b-ADPL3 construct treated with 0.1 mM IPTG was dissolved in PBS and sonicated for 5 min. Cell lysates were centrifuged at 15,000 rpm for 30 min. The supernatant was saved for affinity column chromatography using Ni²⁺-NTA agarose (Qiagen Company, Westburg, Leusden, Netherlands). Briefly, the supernatant was incubated with Ni²⁺-NTA agarose (0.5 ml for 200 ml E. coli culture) for 2 h at 4° C. and the lysate-resin mixture was loaded onto empty column. After washing twice with wash solution (1× PBS, 20 mM imidazole, pH7.2), ADPL3 protein was eluted with elution solution (1× PBS, 200 mM imidazole, pH7.2).

### Example 9

### ELISA of CF2K Protein

In this example, an ELISA method was used for the detection of anti CF2K-antibody in the sera of patients with various rheumatic diseases, including SLE, or those of healthy individuals. In this method, 200 ng of purified recombinant CF2K protein was added to each well of Nunc™ ELISA immunoplate. Incubation continued at 4° C. for 16 h. After incubation, protein was washed and 1% BSA (150 μl) was added to each well and incubation continued for 2 h. After incubation, each well was washed with PBS buffer. Each serum of healthy individual and that of rheumatic patient was diluted (1:200) with solution containing 1% BSA. Incubation continued for 60 min. After reaction, washing with PBS was followed. After washing, goat anti-human IgG-peroxidase was added to each well and incubation continued for 60 min. After reaction, each well was washed with PBS/0.1% Tween 20. Subsequently, substrate containing tetramethyl benzidine was added and incubation continued for 6 min. Finally, 1 M phosphoric acid (100 μl) was added to each well and the optical density was measured at 450 nm.

### Example 10

### ELISA of ADPNF Protein

In this example, an ELISA method was used for the detection of anti ADPNF-antibody in the sera of patients with various rheumatic diseases, including SLE, or those of healthy individuals. In this method, 200 ng of purified recombinant ADPNF protein was added to each well of Nunc™ ELISA immunoplate. Incubation continued at 4° C. for 16 h. After incubation, protein was washed and 1% BSA (150 μl) was added to each well and incubation continued for 2 h. After incubation, each well was washed with PBS buffer. Each serum of healthy individual and that of rheumatic patient was diluted (1:200) with solution containing 1% BSA. Incubation continued for 60 min. After reaction, washing with PBS was followed. After washing, goat anti-human Ig G-peroxidase was added to each well and incubation continued for 60 min. After reaction, each well was washed with PBS/0.1% Tween 20. Subsequently, substrate containing tetramethyl benzidine was added and incubation continued for 6 min. Finally, 1 M phosphoric acid (100 μl) was added to each well and the optical density was measured at 450 nm.

### Example 11

### ELISA of ADPL2 Protein

In this example, an ELISA method was used for the detection of anti ADPL2-antibody in the sera of patients with various rheumatic diseases, including SLE, or those of healthy individuals. In this method, 200 ng of purified recombinant ADPL2 protein was added to each well of Nunc™ ELISA immunoplate. Incubation continued at 4° C. for 16 h. After incubation, protein was washed and 1% BSA (150 μl) was added to each well and incubation continued for 2 h. After incubation, each well was washed with PBS buffer. Each serum of healthy individual and that of rheumatic patient was diluted (1:200) with solution containing 1% BSA. Incubation continued for 60 min. After reaction, washing with PBS was followed. After washing, goat anti-human IgG-peroxidase was added to each well and incubation continued for 60 min. After reaction, each well was washed with PBS/0.1% Tween 20. Subsequently, substrate containing tetramethyl benzidine was added and incubation continued for 6 min. Finally, 1 M phosphoric acid (100 μl) was added to each well and the optical density was measured at 450 nm.

### Example 12

### ELISA of ADPL3 Protein

In this example, an ELISA method was used for the detection of anti ADPL3-antibody in the sera of patients with various rheumatic diseases, including SLE, or those of healthy individuals. In this method, 200 ng of purified recombinant ADPL3 protein was added to each well of nunc ELISA immunoplate. Incubation continued at 4° C. for 16 h. After incubation, protein was washed and 1% BSA (150 μl) was added to each well and incubation continued for 2 h. After incubation, each well was washed with PBS buffer. Each serum of healthy individual and that of rheumatic patient was diluted (1:200) with solution containing 1% BSA. Incubation continued for 60 min. After reaction, washing with PBS was followed. After washing, goat anti-human IgG-peroxidase was added to each well and incubation continued for 60 min. After reaction, each well was washed with PBS/0.1% Tween 20. Subsequently, substrate containing tetramethyl benzidine was added and incubation continued for 6 min. Finally, 1 M phosphoric acid (100 μl) was added to each well and the optical density was measured at 450 nm.

### Example 13

### ELISA of Sm Protein

ELISA employing Sm protein was carried out according to the instruction manual provided by the manufacturer (BL Diagnostika). We used commercially available diagnostic kit for the detection of SLE provided by the manufacturer. In this example, an ELISA method was used for the detection of anti Sm-antibody in the sera of patients with SLE or those of healthy individuals. In this, each well was coated with recombinant purified Sm protein. Each serum of healthy individual and that of patient with SLE was diluted (1:200) with solution containing 1% BSA. Incubation continued for 30 min. After reaction, washing with 300 μl of washing solution was followed. After washing, 100 μl of polyclonal rabbit anti-human IgG-peroxidase was added to each well and incubation continued for 15 min. After reaction, each well was washed three times with 300 μl of washing solution. Subsequently, 100 μl of substrate containing tetramethyl benzidine was added and incubation continued for 15 min. Finally, 1 M phosphoric acid (100 μl) was added to each well and the optical density was measured at 450 nm. According to the instruction manual, positive reactivity is defined when the value is >25 units/ml.

Applications

In this invention, PARP polypeptides, such as but not limited to CF2K and L3, showed high specificity and sensitivity for diagnosis of systemic lupus erythematosus (SLE). This suggests that PARP polypeptide is an important SLE serologic marker in the laboratory testing for the presence of SLE. Patient serum can be screened against cloned protein to identify sera with anti-PARP polypeptide antibodies. This screening can be done using Western immunoblot, ELISA assays or by binding the antigen to microspheres and identifying reactive sera with flow cytometry. These techniques allow for a quick and more reliable diagnosis of SLE patients that might otherwise be diagnosed with another disease presenting similar symptoms.

In one embodiment, using PARP polypeptide allows the characterization of a distinct SLE afflicted cohort of patients that would not have been otherwise identifiable as SLE due to lack of reactivity towards Sm or dsDNA or SR. The production of PARP polypeptide can be scaled for clinical purposes by using known genetic engineering techniques to construct an expression vector such as a plasmid carrying the cDNA sequence encoding PARP polypeptide ligated into it. The expression vector carrying PARP polypeptide can be transfected into various host cells. Host cells range from bacteria to plant or animal cells. The PARP polypeptide cDNA sequence can be engineered into other vectors, such as virus, for insertion into a host organism. The host, whether it is a bacteria, virus, plant or animal, is then the product of the gene expression vector. The production of PARP polypeptide would be useful in the production of antibodies directed to PARP polypeptide cDNA sequence. The antibodies to PARP polypeptide are useful in competition ELISA protocols, therapeutically against SLE, or as reporter molecules in conjunction with the PARP polypeptide itself. The production of PARP polypeptide would be useful for the development of diagnostic kit for the detection of systemic lupus erythematosus. This kit would contain PARP polypeptide antigen bound to solid support, secondary antibody preparation containing detectable label, a buffer, a substrate, and an instruction manual. In this kit, solid support would be a microtiter plate, polystyrene bead, test tube or dip stick. In this kit, a detectable label is a radiolabel, an enzyme, biotin, a dye, a fluorescent tag label, or a luminescent label. In this kit, an enzyme may be alkaline phosphatase or horseradish peroxidase. In this kit, a substrate may contain but not limited to tetramethyl benzidine.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method of diagnosing an autoimmune disease in a mammal comprising: a) obtaining an antibody-containing sample from a subject suspected of suffering from the autoimmune disease, b) contacting said sample with a composition comprising PARP polypeptide; and c) detecting the presence of PARP polypeptide antigen/anti-PARP polypeptide antibody complex, which is indicative of the presence of the autoimmune disease.

2. The method of claim 1, wherein said autoimmune disease is systemic lupus erythematosus.

3. The method of claim 2, wherein the PARP polypeptide has 70% identity to ADPCF2K or ADPL3.

4. The method of claim 1, wherein said sample is blood, plasma or serum.

5. The method of claim 1, wherein said detecting comprises a technique selected from the group consisting of ELISA, RIA, immunoprecipitation, immunofluorescence, and Western immunoblot.

6. The method of claim 5, wherein detecting is carried out by labeled antibody.

7. The method of claim 6, wherein the label is selected from the group consisting of a radiolabel, enzyme, biotin, a dye, a fluorescent tag, a hapten, and a luminescent label.

8. The method of claim 5, wherein ELISA comprises the steps of: a) providing a preparation comprising a PARP polypeptide antigen bound to a support; b) contacting said preparation with said sample whereby PARP polypeptide antigen/anti-PARP polypeptide antibody complex is formed: and c) contacting said complex with a detection agent.

9. The method of claim 8, wherein said detection agent is an anti-Fc antibody that binds said anti-PARP polypeptide antibody.

10. The method of claim 9, wherein said antibody is labeled with a label selected from the group consisting of a radiolabel, an enzyme, biotin, a dye, a fluorescent tag label, and a luminescent label.

11. The method of claim 8, wherein said support is a microtiter plate, polystyrene bead, test tube or dip stick.

12. A diagonistic kit comprising: a) PARP polypeptide antigen preparation; b) a suitable container therefor; and c) instructions for its use to diagnose autoimmune disease.

13. The kit of claim 12, further comprising a second antibody preparation.

14. The kit of claim 13, wherein said second antibody preparation comprises a detectable label.

15. The kit of claim 12, wherein said PARP polypeptide preparation is bound to a support.

16. The kit of claim 15, wherein said support is a microtiter plate, polystyrene bead, test tube or dip stick.

17. The kit of claim 14, wherein said detectable label is a radiolabel, an enzyme, biotin, a dye, a fluorescent tag label, or a luminescent label.

18. The kit of claim 17, wherein said enzyme is alkaline phosphatase or horseradish peroxidase.

19. The kit of claim 12, further comprising a) a buffer or diluent; and b) a suitable container therefor.

20. A method of diagnosing an autoimmune disease in an Asian person comprising: a) obtaining an antibody-containing sample from a subject suspected of suffering from the autoimmune disease, b) contacting said sample with a composition comprising PARP polypeptide; and c) detecting the presence of PARP polypeptide antigen/anti-PARP polypeptide antibody complex, which is indicative of the presence of the autoimmune disease.
